(19) United States
(12) Patent Application Publication
(10) Pub. No.: US 2025/0222157 A1
(43) Pub. Date: Jul. 10, 2025

(54) STERILIZATION DEVICE

(71) Applicant: INNOCIPLE CO., LTD., Hanam-si (KR)

(72) Inventor: Jin Hong AHN, Namyangju-si (KR)

(21) Appl. No.: 19/094,990

(22) Filed: Mar. 30, 2025

Related U.S. Application Data
(63) Continuation of application No. PCT/KR2022/014717, filed on Sep. 30, 2022.

(51) Int. Cl. A61L 9/14

(52) U.S. Cl. CPC A61L 9/14; A61L 2209/134

(57) ABSTRACT

Proposed is a sterilization device. The sterilization device includes a housing in which a liquid-phase base solution is accommodated, an electrode unit spaced mounted inside the housing by being spaced apart from a surface of the base solution and configured to receive a voltage, and a spraying unit spaced apart from the electrode unit and provided with a plate in which at least one coupling hole is formed, the spraying unit being provided with at least one nozzle which is coupled to the coupling hole and which protrudes through an upper surface and a lower surface of the plate. By performing electrostatic spraying, bacteria and bad odors in the air are capable of being removed.

### TECHNICAL FIELD

The present disclosure relates to a sterilization device. More particularly, the present disclosure relates to a sterilization device capable of removing bacteria and bad odors in the air by using electrostatic spraying.

### BACKGROUND ART

Generally, a major factor determining the quality of an indoor environment is maintaining a pleasant air quality. However, due to industrialization and urbanization, various types of harmful substances are contained in an indoor space or a closed space, so that the health of many people is threatened as they spend time in a state in which they are exposed to these harmful substances.

In addition, recently, since Volatile Organic Chemicals (VOCs) included in building materials have been found to be a major cause of the sick house syndrome diseases such as atopic dermatitis or allergic symptoms, various efforts have been made to maintain a pleasant indoor environment. Particularly, due to an increase in the risk of infection that can be seen in the recent increase in atopy, asthma, and allergic symptoms, an explosive outbreak of a new infectious disease, and so on, a demand for air quality control in the indoor environment such as sterilization and deodorization is increasing.

In order to keep the air in the indoor environment in a hygienic and safe manner, it is very important to remove harmful substances contained in the indoor air and to sterilize and deodorize the indoor air. Therefore, various air conditioner products such as air conditioners that include the removal, sterilization, and deodorization functions of harmful substances, and spray-type odor removers having sterilization and deodorization functions are being developed.

In order to escape from such a harmful environment, conventionally, a method of treating and purifying the air for increasing the air quality of the indoor environment in various ways is used. However, most of the conventional air purification methods use a filter method. That is, most of the conventional air conditioners having sterilization and deodorization functions filter out harmful substances in air by using a filter mounted inside the air conditioners.

However, since a large amount of harmful substances that are not filtered by a filter may be included in the indoor air and the life of a filter device and the performance of sterilization and deodorization by the filter are also limited, the conventional air conditioner does not exert sterilization and deodorization effects as much as consumers expect. That is, in a filter-type air purification system used in the conventional air conditioner, the maintenance cost such as periodic replacement of the filter is increased, dust collection performance decreases as the use time passes, and noise is generated during the process of passing air through the filter.

In the conventional air conditioner, moisture is condensed on a surface of a heat exchanger due to a temperature difference between the temperature of the surface of the heat exchanger mounted inside the air conditioner and the temperature of the air. Moisture condensed on the surface of the heat exchanger creates an environment suitable for various fungi and bacteria to breed while the air conditioner is not operated. Eventually, when the air conditioner is not operated for a long time, bad odor is generated by fungi and bacteria that are propagated inside the air conditioner, and the indoor air is further contaminated.

In order to solve this problem, when the spray-type odor remover having the sterilization and deodorization functions is used, there is a hassle that a user is required to spray a deodorant directly from time to time. In addition, when the user sprays the deodorant, there is an inconvenience of using the deodorant due to an irritating strong smell of the deodorant.

Therefore, as a mechanism for enabling sterilization or deodorization, efforts to attempt the sterilization or deodorization effect by using an electrostatic spraying method have recently been increased. The electrostatic spraying method is a technology in which an electric field having a high intensity is applied to a solution supplied to a fine nozzle and the solution is atomized by using a surface change characteristic of the solution. In the electrostatic spraying, various spraying characteristics exist due to an applied voltage, a flow rate, a surface tension of the solution, an electrical conductivity, a supply flow rate, and so on.

A conventional electrostatic spray system includes an electrode part, a blowing part, and a micro-sized droplet supplying part. The electrode part has a pair of electrodes, and a predetermined voltage is applied between the electrodes, so that water vapor or micro-sized droplets are ionized through a plasma discharge and then are discharged to the outside. The blowing part is provided outside the electrode part and forcibly provides a circulating air toward the electrode part. The micro-sized droplet supplying part is configured as a mist generator configured to supply micro-sized droplets between the electrode part and the blowing part.

As such, according to the conventional electrostatic spraying system, fluid provided as the micro-sized droplets by the micro-sized droplet supplying part is transferred toward the electrode part through the blowing part, and the transferred fluid is discharged to the air or to the outside by the electrode part. That is, in the conventional electrostatic spraying system, the micro-sized droplets are generated primarily by the micro-sized droplet supplying part, is transferred to the electrode part, and then is discharged secondarily as a mist ionized by a discharge action.

However, in the conventional electrostatic spraying system, there is a problem that interference occurs between the nozzles positioned adjacent to each other, so that it is difficult to perform a mass production of the conventional electrostatic spraying system.

In addition, in the conventional electrostatic spraying system, since the ionized mist is finally discharged through several processes, there is a problem that there are many elements for performing each process, so that the device is complicated and it is difficult to manage the device. Particularly, in the conventional electrostatic spraying system, since the micro-sized droplets are required to be transferred toward the electrode part through a separate blowing part in a state in which the micro-sized droplets are generated by the micro-sized droplet supplying part, the corresponding configurations are required to be provided in one device, so that there is a problem that the size of the device is increased.

That is, in the conventional electrostatic spraying system, in addition to the electrode part that generates the mist so as to provide direct sterilization or deodorization effects, the micro-sized droplet supplying part and the blowing part are required to be separately provided, so that there is a problem that the management and the maintenance of these configurations are required to be performed.

In addition, in the past, in addition to the aforementioned atomization-type device, there is a problem that it is difficult to perform an electrostatic spraying action since a separate supply pump or the like is used so as to transfer fluid toward an electrostatic spraying device or fluid is transferred toward the electrostatic spraying device by using a mechanical and structural action.

The foregoing is intended merely to aid in the understanding of the background of the present disclosure, and is not intended to mean that the present disclosure falls within the purview of the related art that is already known to those skilled in the art.

### Patent Document

(Patent Document 1) Korean Patent No. 10-1860719 (May 17, 2018)

### DISCLOSURE

### Technical Problem

Accordingly, the present disclosure has been made keeping in mind the above problems occurring in the related art, and an objective of the present disclosure is to provide a technology for providing a sterilization device configured such that a liquid-phase base solution absorbed through an absorption member is converted into a discharge solution by using an electric field and the discharge solution is discharged through a tip part of the absorption member or a liquid-phase solution atomized by an atomization unit is attached to an electrostatic spray unit and forms the discharge solution having a high charge and being in a nano-sized droplet state by using an electric force, the sterilization device being configured such that the discharge solution having hydroxide ions (OH⁻) is discharged by the electric field generated due to a voltage difference between a voltage application device and a discharge part to which the droplets are discharged, thereby being capable of performing a sterilization function and air purification function by simply using the discharge solution without using a separate deodorant or a separate air conditioner.

Meanwhile, another objective of the present disclosure is to provide a sterilization device capable of performing an electrostatic spraying operation by supplying a base solution.

In addition, still another objective of the present disclosure is to provide a sterilization device capable of preventing the spraying inefficiency due to interference caused between adjacent nozzles.

### Technical Solution

In order to achieve the objectives described above, according to an aspect of the present disclosure, there is provided a sterilization device including: an electrostatic spray unit configured to convert a liquid-phase base solution stored in a housing into a discharge solution having a sterilization function using hydroxide ions; and a discharge guide unit coupled to a first side of the housing and provided with a discharge flow path that guides the discharge solution converted by the electrostatic spray unit to be discharged outside the housing.

The sterilization device may further include: an atomization unit provided inside the housing and configured to generate the discharge solution by atomizing the base solution, wherein the electrostatic spray unit is positioned above or below the atomization unit, and is configured to convert the discharge solution generated by the atomization unit into a micro-sized droplet state having a high charge.

The electrostatic spray unit may include: an electrode plate mounted in the discharge flow path and provided with a through-hole through which the discharge solution is capable of passing; and an electrode module provided with an electrode pin, the electrode module being disposed in the discharge flow path such that the electrode module is spaced apart from the electrode plate and faces the electrode plate,
wherein the electrostatic spray unit may be configured to convert the discharge solution which is atomized by the atomization unit and which passes through the discharge flow path into the micro-sized droplet state having the high charge and having the sterilization function by using an electric field generated between the electrode plate and the electrode pin.

The discharge guide unit may include: a dispersion tube part provided with a dispersion tube part body coupled to the housing such that the dispersion tube part body is positioned below the electrode plate, a shielding part disposed inside the dispersion tube part body, and a plurality of dispersion tube part passages formed on a border of the shielding part such that the discharge solution scattered by the atomization unit is dispersed and flows toward the electrode plate; and a retainer positioned above the electrode plate, the retainer being provided with a retainer body having an inner side thereof provided with a retainer passage through which the discharge solution is capable of passing, and the retainer being provided with a support stand disposed inside the retainer body such that the support stand is connected to the retainer body,
wherein the discharge guide unit may be configured such that the discharge solution generated by the atomization unit is guided toward the electrostatic spray unit.

The sterilization device may further include: a support plate disposed above the dispersion tube part, the support plate having an inner side thereof being provided with a support plate passage through which the discharge solution is capable of passing, and the support plate being provided with a pedestal part which is disposed around the support plate passage and which supports the electrode plate,
wherein the pedestal part may be formed in a shape covering the plurality of dispersion tube part passages when the pedestal part is viewed from above or below the pedestal part.

The sterilization device may further include: a blower for blowing air to the discharge flow path,
wherein an air flow path through which air blown from the blower is capable of passing may be provided inside the dispersion tube part, and a plurality of spraying ports connected to the air flow path such that air is capable of being sprayed toward the electrode plate may be formed in the shielding part, and
wherein the plurality of spraying ports may be disposed obliquely toward the electrode plate such that a swirl airflow containing the discharge solution is capable of being formed by spraying air in an inclined direction with respect to the electrode plate.

The discharge guide unit may include: a dispersion tube part coupled to the housing such that the dispersion tube part is positioned below the electrode plate, the dispersion tube part being provided with a plurality of dispersion tube part passages configured to disperse the discharge solution scattered by the atomization unit and to move the discharge solution toward the electrode plate; and a retainer positioned above the electrode plate, the retainer being provided with a retainer body having an inner side thereof provided with a retainer passage through which the discharge solution is capable of passing, and the retainer being provided with a support stand disposed inside the retainer body such that the support stand is connected to the retainer body,
wherein the support stand may include a spoke that extends toward a center side of the retainer passage from an inner circumferential surface of the retainer body, and may include a hub which is connected to the spoke such that the hub is positioned at a center of the retainer passage and which has an inner side provided with a coupling hole,
wherein a voltage supply device mounted in the housing so as to supply a voltage to the electrode plate and the electrode pin may further be provided, and
wherein respective wire passages through which a wire for supplying the voltage of the voltage supply device is capable of passing may be formed in the dispersion tube part, the retainer body, and the spoke.

The electrostatic spray unit may include: a first electrode module provided inside the housing and provided with a plurality of discharge holes through which the discharge solution is discharged; and a second electrode module disposed such that the second electrode module is spaced apart from the first electrode module, the second electrode module being configured to transfer the liquid-phase base solution stored inside the housing to a position corresponding to the plurality of discharge holes of the first electrode module and to convert the base solution into the discharge solution by a potential difference generated between the first electrode module and the second electrode module.

The second electrode module may be a base solution absorption electrode module configured such that a lower end portion of the base solution absorption electrode module absorbs the liquid-phase base solution stored in a base solution storage chamber of the housing and a tip part is formed on an end portion of the base solution absorption electrode module so that the absorbed liquid-phase base solution is capable of being sprayed therethrough,
wherein the first electrode module may be an electrode plate module which is mounted inside the housing such that the electrode plate module is spaced apart from the tip part of the base solution absorption electrode module and which has a discharge hole corresponding to the tip part, and
wherein the base solution absorbed into the base solution absorption electrode module may be converted into the discharge solution in a micro-sized droplet state having a high charge and having the sterilization function by an electric field generated between the tip part and the electrode plate module, and the discharge solution may pass through the discharge hole.

The second electrode module may be a base solution absorption electrode module configured such that a lower end portion of the base solution absorption electrode module absorbs the liquid-phase base solution stored in a base solution storage chamber of the housing and a tip part is formed on an upper end portion of the base solution absorption electrode module so that the absorbed liquid-phase base solution is capable of being sprayed therethrough,
wherein the base solution absorption electrode module may include: a support cover having a side surface provided with a base solution inlet opening part such that the liquid-phase base solution stored in the base solution storage chamber is introduced through the base solution inlet opening part; and an absorption member seated and supported inside the support cover and configured to absorb the liquid-phase base solution introduced into an internal space of the support cover, and wherein the tip part may be formed on an upper end portion of the absorption member.

The housing may include: a storage casing formed in a container shape with an open upper surface, the storage casing having an inner portion thereof provided with the base solution storage chamber in which the liquid-phase base solution is capable of being stored; and an operation body coupled to the open upper surface of the storage casing such that the operation body is capable of being attached to and detached from the open upper surface of the storage casing, the operation body having an inner portion provided with a blowing chamber that is separated and partitioned from the base solution storage chamber, and the operation body having an upper surface thereof provided with a blowing hole and having a lower surface thereof provided with a coupling hole,
wherein the support cover may be formed in a container shape with an open upper surface, and an upper end of the support cover may be coupled to an outer circumference portion of the coupling hole of the operation body.

A plurality of protrusion ribs may be formed on an inner side surface of the support cover such that a separation distance is maintained between the inner side surface of the support cover and an outer side surface of the absorption member and the protrusion ribs are spaced apart from each other in a circumferential direction so that the protrusion ribs press and support the outer side surface of the absorption member.

The sterilization device may further include: a voltage application unit configured to apply a voltage between the base solution absorption electrode module and the electrode plate module,
wherein the voltage application unit may include: a needle electrode which protrudes downward or upward on the operation body and which is coupled to the operation body such that the needle electrode is in contact with the liquid-phase base solution stored in the base solution storage chamber; and a voltage supply device configured to supply a voltage to the electrode plate module and the needle electrode such that a potential difference occurs between the electrode plate module and the liquid-phase base solution in the base solution storage chamber.

A lower operating limit where an operation of the voltage application unit is stopped may be formed at a position corresponding to a lower end portion of the needle electrode or the lower end portion of the base solution absorption electrode module, and
wherein the voltage application unit may be configured to stop applying the voltage between the base solution absorption electrode module and the electrode plate module when a level of the liquid-phase base solution stored in the base solution storage chamber of the housing is equal to or lower than a level of the lower operating limit.

A blowing fan configured to blow air toward a blowing hole may be mounted in a blowing chamber of an operation body, and the discharge solution in a micro-sized droplet state having a high charge and having the sterilization function by passing through a discharge hole of an electrode plate module may be discharged outside through the blowing hole according to a flow of air by the blowing fan, and
wherein an air inlet hole may be formed on a side surface of the operation body such that external air is capable of being introduced into the blowing chamber, and an air filter may be mounted inside the side surface of the operation body such that external air introduced through the air inlet hole passes through the air filter.

In order to achieve the objectives described above, according to another aspect of the present disclosure, there is provided a sterilization device including: an electrostatic spray unit configured to convert a liquid-phase base solution stored in a housing into a discharge solution having a sterilization function using hydroxide ions; and a height adjustment unit provided inside the housing, the height adjustment unit being configured such that a height of the electrostatic spray unit is automatically adjusted according to a storage boundary height of the base solution.

The height adjustment unit for adjusting the height of the electrostatic spray unit may be a floating unit which floats on the base solution and which is adjusted by the base solution, wherein the floating unit may further include a discharge guide unit configured to absorb the base solution and to convert the base solution into the discharge solution having the sterilization function using hydroxide ions (OH⁻), the discharge guide unit being configured to guide the discharge solution to be discharged outside the housing.

In order to achieve the objectives described above, according to still another aspect of the present disclosure, there is provided a sterilization device including: a housing that stores a liquid-phase base solution therein; and a spraying unit configured to spray a spraying solution in which a plurality of heterogeneous ions carrying an electrical charge by applying a high-voltage to the base solution is included and has an airborne bacteria sterilization power of 90% or more at an air temperature of 25 degrees Celsius and an air velocity of 2 m/s.

In order to achieve the objectives described above, according to an embodiment of the present disclosure, a sterilization solution may include: a plurality of heterogeneous ions carrying an electrical charge; and a spraying solution having an airborne bacteria sterilization power of 90% or more at an air temperature of 25 degrees Celsius and an air velocity of 2 m/s.

The plurality of heterogeneous ions may include at least one ion selected from H⁺, OH⁻, and O₂⁻.

In order to achieve the objectives described above, according to an aspect of the present disclosure, there is provided an array module for electrostatic spraying configured to convert a liquid-phase base solution into micro-sized droplets by generating static electricity between electrodes as power is applied to the array module, the array module including: a plate formed in a plate shape and provided with at least one coupling hole; and at least one nozzle coupled to the coupling hole and provided such that both end portions of the nozzle protrude through an upper surface and a lower surface of the plate.

In addition, the nozzle may include: an absorption part which protrudes through the lower surface of the plate and which is immersed in the liquid-phase base solution; and a tip part which extends from the absorption part and which protrudes through the upper surface of the plate.

In addition, the nozzle may further include: a cone part which protrudes on an outer side of the tip part and which extends obliquely and outwardly to the upper surface of the plate from an end portion of the tip part; and a hole part formed such that the hole part penetrates the nozzle and the hole part is in communication with the coupling hole.

Meanwhile, the nozzle may include: at least one support tube which is coupled to the coupling hole and which protrudes through the upper surface and the lower surface of the plate; and at least one absorption member which is inserted into a tube of the support tube and which has an end portion that extends such that the end portion of the absorption member is immersed in the liquid-phase base solution. Here, the absorption member may include a fiber bundle in which a plurality of fibers is coupled to each other.

In addition, the nozzle may further include a porous cap provided on the support tube and configured to partition the fibers included in the fiber bundle such that the fibers are divided in a single state or in a state in which the fibers are divided into two or more fibers.

In addition, the porous cap of the nozzle may include a first cap member coupled to a first side end of the support tube and a second cap member which faces the first cap member and which is coupled to a second side end of the support tube, and wherein the nozzle may further include a gap maintaining member which is disposed between the first cap member and the second cap member and which is coupled to an inner side of the support tube so as to surround the fiber bundle.

In addition, the nozzle may further include an expansion prevention cover which is coupled to a first side end of the support tube and which supports the fiber bundle. Here, the expansion prevention cover may be formed in a shape which is wider at a lower side thereof and narrower at an upper side thereof on the first side end of the support tube, and may be configured to press an end portion of the fiber bundle from outside.

In addition, a unit fiber including a plurality of unit fibers is capable of being coupled to the fiber bundle, and the unit fiber may be selected from a group consisting of glass fiber, carbon fiber, metal fiber, aramid fiber, and a mixture or combination thereof.

Meanwhile, the array module for electrostatic spraying according to an aspect of the present disclosure may further include at least one micro protrusion which is disposed around the coupling hole and which protrudes through the upper surface of the plate.

In addition, the micro protrusion may include: a first protrusion part forming a group which is spaced apart from a circumference of the coupling hole and which is distributed adjacent to the nozzle; and a second protrusion part forming a group which is spaced apart from a circumference of the first protrusion part and which is distributed at a position far from the nozzle, wherein a length of the first protrusion part may be larger than a length of the second protrusion part.

In order to achieve the objectives described above, according to an aspect of the present disclosure, there is provided a sterilization device including: a housing in which a liquid-phase base solution is accommodated therein; an electrode unit spaced apart from a surface of the base solution and mounted inside the housing, the electrode unit being configured to receive a voltage; and a spraying unit including a plate which is disposed such that the plate is spaced apart from the electrode unit and which has at least one coupling hole, the spraying unit including at least one nozzle which is coupled to the coupling hole and which protrudes through an upper surface and a lower surface of the plate. The electrode unit may further include a power supply mechanism, and the power supply mechanism may be connected to the base solution stored in the housing.

In addition, the nozzle may include: an absorption part which protrudes through the lower surface of the plate and which is immersed in the liquid-phase base solution; and a tip part which extends from the absorption part and which protrudes through the upper surface of the plate.

In addition, the nozzle may include a cone part which protrudes on an outer side of the tip part and which extends obliquely and outwardly to the upper surface of the plate from an end portion of the tip part.

In addition, the nozzle may include: at least one support tube which is coupled to the coupling hole and which protrudes through the upper surface and the lower surface of the plate; and at least one absorption member which is inserted into a tube of the support tube and which has an end portion that extends such that the end portion of the absorption member is immersed in the liquid-phase base solution.

### Advantageous Effects

According to the sterilization device according to an embodiment of the present disclosure, the liquid-phase base solution is atomized by the atomization unit and the electrostatic spray unit sprays the discharge solution in the micro-sized droplet state having hydroxide ions (OH⁻) by using the electric field, so that the sterilization function and the air purification function are capable of being performed by simply using the liquid-phase base solution without using the separate deodorant or the air conditioner.

In addition, since the atomization unit and the electrostatic spray unit are provided together, the atomized discharge solution is generated by atomizing the liquid-phase base solution primarily, and the nano-sized droplets having the high charge are secondarily generated from the atomized discharge solution by using the electrostatic spray unit, so that the discharge solution having the sterilization function using hydroxide ions (OH⁻) is generated. Therefore, the sterilization device of the present disclosure is capable of being more rapidly and efficiently discharging the discharge solution compared to the conventional technology in which electrolysis is directly performed on the liquid-phase base solution so as to convert the liquid-phase base solution into the discharge solution.

According to another aspect of the present disclosure, by absorbing the liquid-phase base solution into the absorption member and spraying the discharge solution from the tip part of the absorption member by the electric field, moisture is electrically ionized, and the discharge solution having hydroxide ions (OH⁻) is sprayed and discharged, thereby performing the sterilization function. Accordingly, the sterilization function is capable of being performed by simply using the liquid-phase base solution without using a separate deodorant or a separate air conditioner. Therefore, in addition to preventing additional contamination of the device, a cleaner, more hygienic, and convenient use of the device is capable of being realized since an irritating strong smell is not generated and a periodic operation by the user is not required.

In addition, since the electrode plate module is separated and partitioned from the base solution storage chamber and is stably coupled and fixed to the internal space of the operation body, the separation distance between the electrode plate module and the tip part of the absorption member is capable of being maintained constantly. Through this, a stable electric field in an optimal state is generated between the tip part and the electrode plate module, and stable spraying of the discharge solution is capable of being realized, so that there is an effect that the sterilization function is capable of being performed stably and efficiently.

In addition, since contact area between the absorption member and the liquid-phase base solution is increased, the storage amount of the liquid-phase base solution is increased and the spraying amount of the discharge solution sprayed from the tip part is increased, so that there are effects that the spraying of the discharge solution from the plurality of tip parts is stably performed and the operation stability is increased.

In addition, the risk of the occurrence of the electric shock to the user, the discharge of the remaining solution, and so on are prevented when the operation body is separated from the storage casing, so that there are effects that the user's safety is secured during the use process and the convenience of use of the sterilization device is further increased.

Meanwhile, in the sterilization device according to still another aspect of the present disclosure, since the base solution is supplied and the electrostatic spraying operation is performed, a separate pump or configurations of an atomization device or a blowing device are not required, so that the manufacturing cost of the sterilization device may be lowered. Furthermore, there are effects that the device is capable of being miniaturized, the device is easy to manage, and the cost in terms of maintenance is reduced.

In addition, since the sterilization device according to the present disclosure absorbs the base solution by the nozzle that is provided as an integral part of the plate and then the absorbed base solution is electrostatically sprayed through the nozzle, the base solution is capable of being directly sprayed. Furthermore, since interference between the adjacent nozzles is prevented due to the shape of the tip part, the large amount of aerosols may be stably sprayed, so that the electrostatic spraying efficiency may be increased.

### DESCRIPTION OF DRAWINGS

FIG. 1 is a perspective view illustrating a sterilization device according to an embodiment of the present disclosure.

FIG. 2 is a cross-sectional view illustrating the sterilization device according to an embodiment of the present disclosure.

FIG. 3 is an exploded perspective view illustrating the sterilization device according to an embodiment of the present disclosure.

FIG. 4 is a cross-sectional view illustrating a modification example of the sterilization device according to an embodiment of the present disclosure.

FIG. 5 is a conceptual view illustrating a principle of operating the sterilization device illustrated in FIG. 4.

FIG. 6 is a cross-sectional view illustrating another modification example of the sterilization device according to an embodiment of the present disclosure.

FIG. 7 is a plan view illustrating a portion of the sterilization device illustrated in FIG. 6.

FIG. 8 is a cross-sectional view illustrating still another modification example of the sterilization device according to an embodiment of the present disclosure.

FIG. 9 is a plan view illustrating a portion of the sterilization device illustrated in FIG. 8.

FIG. 10 is a perspective view illustrating the sterilization device according to another embodiment of the present disclosure.

FIG. 11 is an exploded perspective view illustrating a detailed configuration of the sterilization device according to another embodiment of the present disclosure.

FIG. 12 is a cross-sectional view illustrating an internal structure of the sterilization device according to another embodiment of the present disclosure.

FIG. 13 is a view illustrating a principle of spraying a discharge solution of the sterilization device according to another embodiment of the present disclosure.

FIG. 14 is a view illustrating a shape of a needle electrode of the sterilization device according to another embodiment of the present disclosure.

FIG. 15 is a view illustrating a principle of spraying a discharge solution according to a modification example of the sterilization device according to another embodiment of the present disclosure.

FIG. 16 is a view illustrating a state in which an operation body of the sterilization device according to another embodiment of the present disclosure is separated.

FIG. 17 is a view illustrating a modification example of the shape of the needle electrode of the sterilization device according to another embodiment of the present disclosure.

FIG. 18 is an exploded perspective view illustrating a configuration of a base solution absorption electrode module of the sterilization device according to another embodiment of the present disclosure.

FIG. 19 is a perspective view illustrating a tip part of the sterilization device according to another embodiment of the present disclosure.

FIG. 20 is a cross-sectional view illustrating a coupling structure of the base solution absorption electrode module of the sterilization device according to another embodiment of the present disclosure.

FIG. 21 is a cross-sectional view illustrating a modification example of the coupling structure of the base solution absorption electrode module of the sterilization device according to another embodiment of the present disclosure.

FIG. 22 is an exploded perspective view illustrating a modification example of the base solution absorption electrode module of the sterilization device according to another embodiment of the present disclosure.

FIG. 23 is a perspective view illustrating the sterilization device according to still another embodiment of the present disclosure.

FIG. 24 is a cross-sectional view illustrating the sterilization device according to still another embodiment of the present disclosure.

FIG. 25 is an enlarged cross-sectional view illustrating a floating unit of the sterilization device according to still another embodiment of the present disclosure.

FIG. 26 is a perspective view schematically illustrating an array module for electrostatic spraying according to an embodiment of the present disclosure.

FIG. 27 is a cross-sectional perspective view illustrating the array module for electrostatic spraying in FIG. 26.

FIG. 28 is a view illustrating a cross-section of the array module for electrostatic spraying according to an embodiment of the present disclosure.

FIG. 29 is a cross-sectional view illustrating a modification example of the array module for electrostatic spraying according to an embodiment of the present disclosure.

FIG. 30 is a cross-sectional view schematically illustrating a modification example of a nozzle of the array module for electrostatic spraying according to an embodiment of the present disclosure.

FIG. 31 and FIG. 32 are views illustrating the array module for electrostatic spraying according to another embodiment of the present disclosure.

FIG. 33 is a view illustrating another modification example of the array module for electrostatic spraying illustrated in FIG. 32.

FIG. 34 and FIG. 35 are views illustrating the array module for electrostatic spraying according to still another embodiment of the present disclosure.

FIG. 36 is a view illustrating the array module for electrostatic spraying according to still another embodiment of the present disclosure.

FIG. 37 and FIG. 38 are views illustrating the array module for electrostatic spraying according to still another embodiment of the present disclosure.

FIG. 39 is a view schematically illustrating the sterilization device according to an embodiment of the present disclosure.

FIG. 40 is a view illustrating a modification example of the sterilization device according to an embodiment of the present disclosure.

FIG. 41 is a view illustrating another modification example of the sterilization device in FIG. 40 according to an embodiment of the present disclosure.

### MODE FOR INVENTION

Specific structural or functional descriptions of the embodiments of the present disclosure disclosed in the present specification are exemplified only for the purpose of describing the embodiments according to the present disclosure, and the embodiments according to the present disclosure may be implemented in various forms, and should not be construed as being limited to the embodiments described in the present specification.

Since the embodiments according to the present disclosure can be modified in various ways and have various forms, specific embodiments are illustrated in the drawings and will be described in detail in the present specification. However, this is not intended to limit the embodiments according to the concept of the present disclosure to a specific form of disclosure, and it should be understood that all changes, equivalents, and substitutes included in the spirit and scope of the present disclosure are included.

Terms such as first and/or second may be used to describe various components, but the components should not be limited by the terms. The above terms are only for the purpose of distinguishing one component from other components, for example, without departing from the scope of the rights according to the concept of the present disclosure, the first component may be referred to as the second component, and similarly the second component may also be referred to as a first component.

When a component is described as being “connected”, “coupled”, or “linked” to another component, that component may be directly connected, coupled, or linked to that other component. However, it should be understood that yet another component between each of the components may be present. In contrast, it should be understood that when a component is referred to as being “directly coupled” or “directly connected” to another component, there are no intervening components present. Furthermore, the terms used herein to describe a relationship between elements, that is, “between”, “directly between”, “adjacent”, or “directly adjacent” should be interpreted in the same manner as those described above.

The terms used in the present specification are only used to describe specific embodiments, and are not intended to limit the present disclosure. As used herein, the singular forms are intended to include the plural forms as well, unless the context clearly indicates otherwise. In the present specification, terms such as “comprise” or “have” are intended to designate the presence of a set feature, number, step, action, component, part, or combination thereof, but it should be understood that the presence or additional possibilities of one or more other features, numbers, steps, actions, components, parts, or combinations thereof are not preliminarily excluded.

Unless otherwise defined, all terms used herein, including technical or scientific terms, have the same meaning as commonly understood by one of ordinary skill in the art to which the present disclosure belongs. Terms such as those defined in a commonly used dictionary should be interpreted as having a meaning consistent with the meaning of the related technology, and should not be interpreted as an ideal or excessively formal meaning unless explicitly defined in the present specification.

Hereinafter, the present disclosure will be described in detail by describing an exemplary embodiment of the present disclosure with reference to the accompanying drawings. The same reference numerals shown in each drawing indicate the same members.

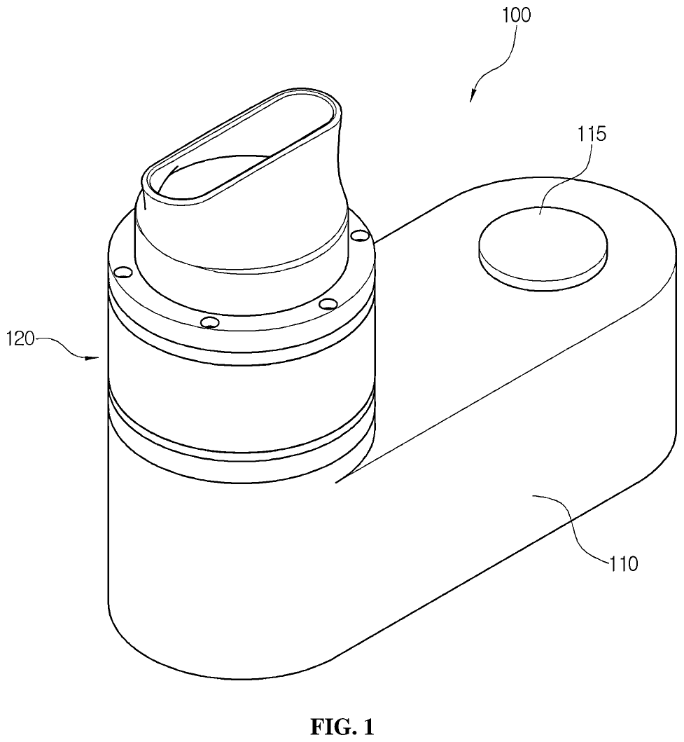

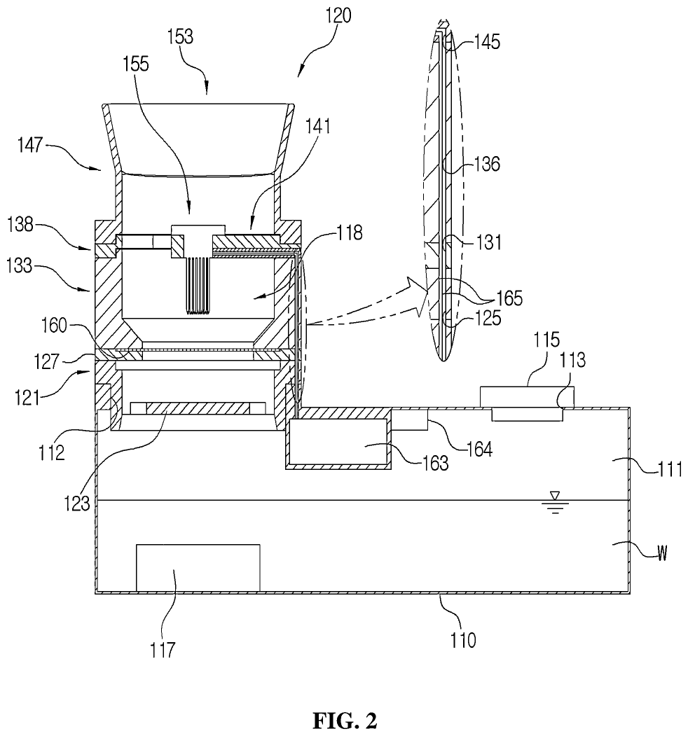

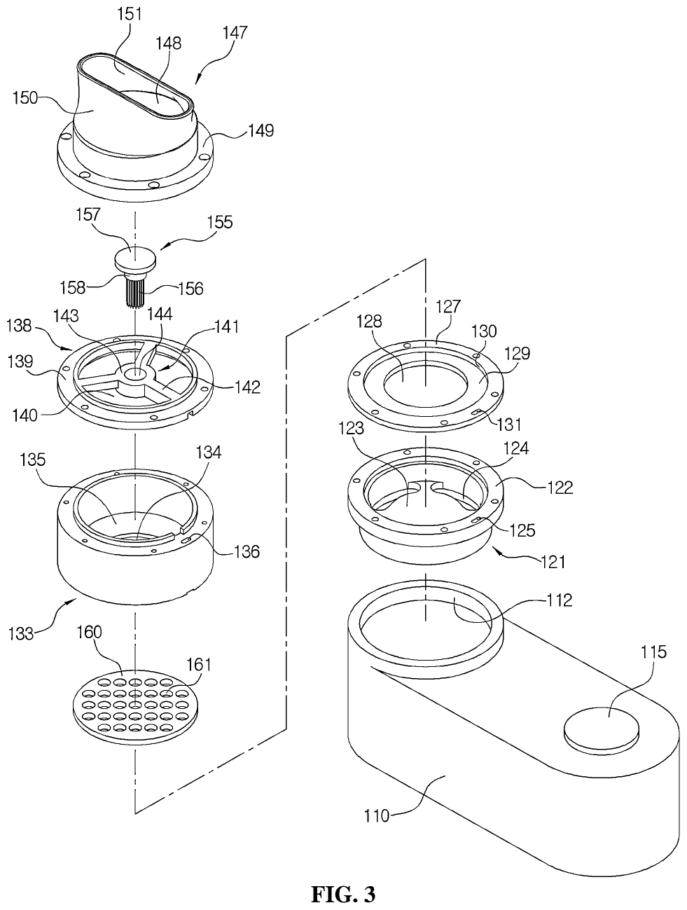

FIG. 1 is a perspective view illustrating a sterilization device according to an embodiment of the present disclosure, FIG. 2 is a cross-sectional view illustrating the sterilization device according to an embodiment of the present disclosure, and FIG. 3 is an exploded perspective view illustrating the sterilization device according to an embodiment of the present disclosure.

The present disclosure relates to a technology of a sterilization device in which bacteria and bad odors in the air are removed by at least one of charges or hydroxide ions (OH⁻) of a discharge solution with a high charge due to an electric field generated by a voltage difference between a voltage applying device and a discharge part to which droplets are discharged.

Referring to FIG. 1 to FIG. 3, a sterilization device 100 according to an embodiment of the present disclosure includes: an electrostatic spray unit 118 configured to convert a discharge solution generated from a base solution stored in a housing 110 that stores the base solution in a liquid-phase into a discharge solution having a sterilization function using hydroxide ions (OH⁻); and a discharge guide unit 120 coupled to a first side of the housing 110, the discharge guide unit 120 providing a discharge flow path 153 that guides the discharge solution converted by the electrostatic spray unit 118 to be discharged to an outside of the housing 110.

The sterilization device 100 according to an embodiment of the present disclosure is applied to an ion generation element, an ion generation apparatus, an air conditioning apparatus, and so on configured to spray a discharge solution containing ions into the indoor air. Particularly, the sterilization device 100 according to an embodiment of the present disclosure may be applied to an air purifier, a humidifier, a dehumidifier, a fan heater, an air conditioner, a refrigerator, and so on. As another embodiment, the sterilization device 100 may be applied to a hair dryer, a pet drying room, or the like configured to supply heated air to hair or body hair, or may be applied to a washing machine, a dryer, or a clothing care apparatus configured to supply heated air to a clothing and so on. As still another embodiment, the sterilization device 100 may be applied to a sterilizer configured to directly spray a discharge solution onto a human or a companion animal for sterilization purposes.

The sterilization device 100 according to an embodiment of the present disclosure may be a portable-type sterilization device in which a power storage device such as a battery or the like is mounted, or may be a stand-type sterilization device connected to a power supply device through a wire or the like.

In the sterilization device 100 according to an embodiment of the present disclosure, an atomization unit 117 may atomize a liquid-phase base solution W contained in the housing 110, the electrostatic spray unit 118 may convert the atomized liquid-phase solution into a high charge discharge solution in a nano-sized droplet state by using an electric field, and the sterilization device 100 may spray the discharge solution forming hydroxide ions (OH⁻) by the electric field generated due to a voltage difference between a power supplying part (not illustrated) and a discharging part to which the droplets are discharged.

That is, by preparing both the atomization unit 117 and the electrostatic spray unit 118, the atomized discharge solution may be generated by atomizing the liquid-phase base solution primarily, and the discharge solution may be converted into the high charge discharge solution in the nano-sized droplet state, the high charge discharge solution having a sterilization function using hydroxide ions (OH⁻). Therefore, the discharge solution may be more rapidly and effectively discharged than that of the conventional technology.

More specifically, the electrostatic spray unit 118 produces ions including O₂⁻(H₂O)n (where n is any natural number) as negative ions and H⁺(H₂O)m (where m is any natural number) as positive ions, and releases these ions into the air, so that bacteria floating in the air is sterilized by an oxidation reaction due to hydrogen peroxide H₂O₂ or OH radicals as an active species generated by a chemical reaction of these ions.

The housing 110 has a chamber 111 in which the liquid-phase base solution W is capable of being stored. An opening 112 connected to the chamber 111 is formed on a first side of the housing 110. The atomized discharge solution may be discharged through the opening 112. An inlet and outlet port 113 is formed on a second side of the housing 110. Through the inlet and outlet port 113, the liquid-phase base solution W may be added to the chamber 111 or discharged from the chamber 111. The inlet and outlet port 113 may be opened and closed by a lid 115.

A specific structure or a specific shape of the housing 110 is not limited to the described structure or the described shape that is illustrated, but may be modified in various ways.

The atomization unit 117 is mounted on an inner side of the housing 110, and may atomize the liquid-phase base solution W stored in the chamber 111. As the atomization unit 117, various types of devices, such as an ultrasonic oscillator, capable of atomizing the liquid-phase base solution W so that the atomized discharge solution is formed may be used. The atomization unit 117 may be operated by receiving power from a power supplying part (not illustrated) mounted in the housing 110. Although it is illustrated in the drawings that the atomization unit 117 is mounted on an inner floor of the housing 110, the mounting position and the mounting number of atomization units 117 may be variously changed.

Here, the power supplying part (not illustrated) may be a charging device such as a battery or a storage battery that supplies power by using a charged electrical energy, or may be a power conversion device connected to a separate domestic or industrial power supply device (for example, an electrical outlet) and configured to supply electrical energy.

The electrostatic spray unit 118 according to an embodiment includes an electrode plate 160 mounted in the discharge flow path 153 and provided with a through-hole 161 through which the discharge solution can pass, and includes an electrode module 155 provided with an electrode pin 156 disposed in the discharge flow path 153 such that the electrode pin 156 is spaced apart from the electrode plate 160 and faces the electrode plate 160. Furthermore, the electrostatic spray unit 118 has a function of converting the discharge solution which is atomized by the atomization unit 117 and which passes through the discharge flow path 153 into the discharge solution having a sterilization function by using an electric field formed between the electrode plate 160 and the electrode pin 156.

The electrode module 155 includes a plurality of electrode pins 156 spaced apart from each other, a support body 157 supporting the plurality of electrode pins 156, and a support boss 158 provided between the support body 157 and the electrode pins 156 and coupled to a first side of the discharge guide unit 120.

The discharge guide unit 120 is coupled to the opening 112 and guides the discharge solution scattered in the chamber 111 to the outside of the housing 110. The discharge flow path 153 through which the discharge solution can pass is provided inside the discharge guide unit 120. The discharge guide unit 120 includes a dispersion tube part 121, a support plate 127, a guide tube part 133, a retainer 138, and a discharge tube part 147.

The discharge guide unit 120 may be coupled to the housing 110 such that the discharge guide unit 120 is capable of being attached to and detached from the housing 110. As an embodiment, the discharge guide unit 120 may be coupled to the housing 110 by a force-fitting manner or a screw coupling manner. As another embodiment the discharge guide unit 120 may be coupled to the housing 110 by a coupling mechanism such as a bolt and so on.

The dispersion tube part 121 is coupled to the housing 110. The dispersion tube part 121 includes a dispersion tube part body 122 inserted into the opening 112 of the housing 110, and includes a shielding part 123 disposed inside the dispersion tube part body 122. A plurality of dispersion tube part passages 124 through which the discharge solution can pass is formed inside the dispersion tube part body 122. The plurality of dispersion tube part passages 124 is disposed on a border of the shielding part 123 along an inner circumference of the dispersion tube part body 122 such that the plurality of dispersion tube part passages 124 is spaced apart from each other. A wire passage 125 through which a wire 165 can pass is formed on an inner side of the dispersion tube part body 122. The wire 165 for supplying a voltage to the electrode module 155 is accommodated in the wire passage 125. Since the wire 165 for supplying the voltage to the electrode module 155 is disposed inside the dispersion tube part body 122, the risk of a safety accident that may occur due to moisture reaching the wire 165 may be reduced, and the sterilization device 100 is capable of being designed in a compact structure.

The dispersion tube part 121 is formed such that an inner center portion of the dispersion tube part body 122 is blocked by the shielding part 123 and the plurality of dispersion tube part passages 124 through which the discharge solution can pass is formed along the circumference of the shielding part 123, so that the discharge solution atomized by the atomization unit 117 may be dispersed and may flow in the dispersion tube part 121. The dispersion tube part 121 may be coupled to the housing 110 such that the dispersion tube part 121 is capable of being attached to and detached from the housing 110 by a coupling manner using a fixing member such as a screw and so on, by a force-fitting manner, or by various other manners.

The specific shape of each of the dispersion tube part body 122 and the shielding part 123 may be variously modified, and the number, the shape, and the position of the dispersion tube part passage 124 may also be variously modified.

The support plate 127 is disposed on the dispersion tube part 121 and supports the electrode plate 160. A support plate passage 128 through which the discharge solution can pass is provided inside the support plate 127, and a pedestal part 129 supporting the electrode plate 160 is provided on a circumference of the support plate passage 128. an accommodating groove 130 on which the electrode plate 160 is accommodated is formed on the pedestal part 129.

More specifically, the pedestal part 129 is formed such that the pedestal part 129 covers the plurality of dispersion tube part passages 124 when viewed from above or below, so that the support plate passage 128 and the plurality of dispersion tube part passages 124 may be disposed in a staggered manner. Accordingly, the flow route and the flow time of the airflow containing the discharge solution may increase as the airflow sequentially passes through the plurality of dispersion tube part passages 124 and the support plate passage 128 that are disposed in the staggered manner.

A wire passage 131 through which the wire 165 can pass is formed on an inner side of the support plate 127. The wire passage 131 is connected to the wire passage 125 of the dispersion tube part 121 such that the wire passage 131 is capable of accommodating the wire 165.

The support plate 127 may be coupled to the dispersion tube part 121 such that the support plate 127 is capable of being attached to and detached from the dispersion tube part 121 by various manners such as a coupling manner using a fixing member such as a screw and so on.

Particularly, since the support plate passage 128 through which the discharge solution can pass is provided inside the support plate 127 so that an inner diameter of the support plate 127 is smaller than an outer diameter of the electrode plate 160, the support plate 127 may support the electrode plate 160 positioned above the support plate 127.

In addition to the structure illustrated in the drawings, the support plate 127 may be modified to various other structures capable of supporting the electrode plate 160 and allowing the discharge solution to pass therethrough. In another embodiment, a support protrusion (not illustrated) which protrudes from a plurality of positions in the support plate 127 to an inner side of the support plate 127 in which the support plate passage 128 is formed and which supports the electrode plate 160 may be formed in the support plate 127, so that the size of the support plate passage 128 may be increased and the flow rate of the flowing discharge solution may be increased.

Here, the electrode plate 160 described above is supported on the support plate 127 such that the electrode plate 160 faces the electrode module 155. A plurality of through-holes 161 is formed in the electrode plate 160 such that the plurality of through-holes 161 penetrates the electrode plate 160 in a thickness direction of the electrode plate 160. The discharge solution passing through the dispersion tube part 121 may flow toward the electrode module 155 by passing through the through-holes 161 of the electrode plate 160. The electrode plate 160 may be supported on the pedestal part 129 by being fully accommodated in the accommodating groove 130 such that an upper surface of the electrode plate 160 does not protrude higher than an upper surface of the support plate 127.

The guide tube part 133 is disposed above the support plate 127 and guides the discharge solution passing through the support plate 127 toward the retainer 138. The guide tube part 133 is disposed between the support plate 127 and the retainer 138, so that there is a distance between the retainer 138 and the electrode plate 160 that is placed on the support plate 127. A guide tube part passage 134 through which the discharge solution passes is provided inside the guide tube part 133. A diffusion guide part 135 formed in a shape in which a width thereof is gradually increased toward the retainer 138 is provided on a circumference of the guide tube part passage 134. The diffusion guide part 135 may diffuse the airflow containing the discharge solution toward the retainer 138, the airflow being formed between the electrode plate 160 and the electrode module 155. A wire passage 136 through which the wire 165 can pass is formed on an inner side of the guide tube part 133. The wire passage 136 is connected to the wire passage 131 of the support plate 127 such that the wire passage 136 is capable of accommodating the wire 165.

The guide tube part 133 may be coupled to the support plate 127 such that the guide tube part 133 is capable of being attached to and detached from the support plate 127 by various manners such as a coupling manner using a fixing member such as a screw and so on. In addition to the structure illustrated in the drawings, the guide tube part 133 may be modified to various other structures capable of supporting the retainer 138 and allowing the discharge solution to pass therethrough while being disposed above the support plate 127.

The retainer 138 is disposed above the guide tube part 133 and supports the electrode module 155. The retainer 138 includes a retainer body 139 coupled to an upper end portion of the guide tube part 133, and includes a support stand 141 disposed inside the retainer body 139 such that the support stand 141 is capable of supporting the electrode module 155. A retainer passage 140 through which the airflow containing the discharge solution can pass is formed inside the retainer body 139. The support stand 141 includes a plurality of spokes 142 that extends from an inner circumferential surface of the retainer body 139 toward a center of the retainer passage 140, and includes a hub 143 supported by the plurality of spokes 142 and positioned at the center of the retainer passage 140.

The electrode module 155 is supported by the hub 143 positioned at the center portion of the retainer 138, and may be fixed to the retainer 138 by being integrally coupled to the hub 143. In addition, the retainer passage 140 of the retainer 138 may be formed around the hub 143 that is positioned at the center portion of the retainer 138, and may be positioned such that the retainer passage 140 and the support plate passage 128 are disposed in a staggered manner. Therefore, the flow route and the flow time are increased as the airflow containing the discharge solution sequentially passes through the plurality of dispersion tube part passages 124, the support plate passage 128, and the retainer passage 140 such that the movement of the airflow is continuously guided to a side direction, so that the time in which the discharge solution is exposed to the electric field formed between the electrode plate 160 and the electrode module 155 may be increased.

A coupling hole 144 that penetrates the hub 143 in the thickness direction of the hub 143 is formed inside the hub 143. A wire passage 145 through which the wire 165 can pass is formed on an inner side of the retainer body 139. The wire passage 145 is connected to the wire passage 136 of the guide tube part 133 such that the wire passage 145 is capable of accommodating the wire 165. The wire passage 145 extends from a first side of the retainer body 139 through the inside of one or more spokes 142 to the hub 143. Therefore, the wire 165 for supplying a voltage to the electrode module 155 may be connected to the electrode module 155 that is coupled to the hub 143 through the wire passage 145 of the retainer 138.

As the wire 165, it is preferable to use a stainless-steel wire covered with a polyfluoride ethylene-based resin that has durability against ozone.

The retainer 138 may be coupled to the guide tube part 133 such that the retainer 138 is capable of being attached to and detached from the guide tube part 133 by various manners such as a coupling manner using a fixing member such as a screw and so on. In addition to the structure illustrated in the drawings, the retainer 138 may be modified to various other structures capable of supporting the electrode plate 155 and allowing the airflow containing the discharge solution to pass through the retainer 138.

The discharge tube part 147 is mounted on the retainer 138 and guides the airflow containing the discharge solution to the outside. A discharge tube part passage 148 through which the airflow containing the discharge solution can pass may be provided inside the discharge tube part 147. The dispersion tube part passage 124 of the dispersion tube part 121, the support plate passage 128 of the support plate 127, the guide tube part passage 134 of the guide tube part 133, the retainer passage 140 of the retainer 138, and the discharge tube part passage 148 form the discharge flow path 153 through which the discharge solution passes.

The discharge tube part 147 includes a discharge tube part body 149 coupled to an upper portion of the retainer 138, and includes a discharge guide 150 disposed on an upper portion of the discharge tube part body 149. A discharge port 151 for discharging the airflow containing the discharge solution passing through the discharge tube part passage 148 is formed on an end of the discharge guide 150. Since the discharge guide 150 is formed in a shape in which the width of the discharge guide 150 gradually decreases in an upward direction, the discharge speed of the airflow containing the discharge solution passing through the discharge tube part passage 148 may be increased, and the discharge solution may be discharged over a longer distance.

In addition, a fan (not illustrated) configured to be operated so as to discharge the airflow containing the discharge solution over a longer distance directly may be further provided in the discharge tube part 147.

The discharge tube part 147 may have a shape in which a cross-sectional surface of a lower portion of the discharge tube part 147 is formed in a circular shape, and the shape of the discharge tube part 147 is narrowed in a linear direction as the discharge tube part 147 extends toward the discharge guide 150. Accordingly, a wind direction may be adjusted by rotation of the discharge tube part 147 and the discharge guide 150. In addition, a plurality of louvers (not illustrated) for adjusting the wind direction may be provided in the discharge guide 150 so as to adjust a direction of the airflow discharged through the discharge guide 150.

In addition, a discharge cover (not illustrated) coupled to the discharge guide 150 such that the discharge cover is capable of being attached to and detached from the discharge guide 150 may be further provided on the discharge guide 150 of the discharge tube part 147. Furthermore, the discharge guide 150 may be closed as the discharge cover (not illustrated) is coupled to the discharge tube part 147 so as to prevent foreign substances and so on to be introduced into the discharge tube part 147 in a state in which the operation of the sterilization device is stopped.

The discharge tube part 147 may be coupled to the retainer 138 such that the discharge tube part 147 is capable of being attached to and detached from the retainer 138 by various manners such as a coupling manner using a fixing member such as a screw and so on. In addition to the structure illustrated in the drawings, the discharge tube part 147 may be modified to various other structures capable of being disposed on the upper portion of the retainer 138 and discharging the airflow containing the discharge solution to the outside.

The discharge guide unit 120 is capable of being separated from the housing 110. Furthermore, the dispersion tube part 121, the support plate 127, the guide tube part 133, the retainer 138, and the discharge tube part 147 forming the discharge guide unit 120 are capable of being separated from each other. As described above, when the discharge guide unit 120 is manufactured in a detachable prefabricated structure, only parts that have abnormalities during use may be replaced, and maintenance is more advantageous. In addition, when the housing 110 is required to be cleaned during use, the housing 110 is capable of being easily cleaned by separating the discharge guide unit 120 from the housing 110.

In addition to the structure illustrated in the drawings, the discharge guide unit 120 may be modified to various other structures capable of supporting the electrode module 155 and the electrode plate 160 and discharging the discharge solution to the outside of the housing 110.

The electrode module 155 is disposed in the discharge flow path 153 of the discharge guide unit 120 such that the electrode module 155 is spaced apart from the electrode plate 160 and faces the electrode plate 160. The plurality of electrode pins 156 is supported on the support body 157 such that the plurality of electrode pins 156 is spaced apart from each other, and is coupled to the retainer 138. The support body 157 includes the support boss 158 inserted into the coupling hole 144 of the retainer 138. The number of electrode pins 156 and the structure of the support body 157 supporting the plurality of electrode pins 156 may be variously modified, and the coupling structure of the electrode pins 156 and the retainer 138 may be variously modified. However, as illustrated in the drawings, the coupling structure in which the support body 157 is inserted into the coupling hole 144 of the retainer 138 may act more advantageously for assembling the electrode module 155 to the retainer 138 or for separating the electrode module 155 from the retainer 138.

The electrode module 155 may receive a voltage supplied from a voltage supply device 163 through the wire 165 disposed in the inner side of the retainer 138. The voltage supply device 163 is mounted in the housing 110, and may supply a voltage to the electrode module 155 and the electrode plate 160. The voltage supply device 163 may be electrically connected to the electrode module 155 and the electrode plate 160 through the wire 165 that is disposed in the inner side of the discharge guide unit 120.

For example, the voltage supply device 163 may supply a + voltage to the electrode module 155, and may supply a − voltage to the electrode plate 160. In another embodiment, the voltage supply device 163 may supply an alternating current power between the electrode plate 160 and the electrode module 155.

In the drawings, it is illustrated that the voltage supply device 163 is mounted inside the housing 110, but the mounting position of the voltage supply device 163 is capable of being modified in various ways.

Since the voltage supply device 163 supplies a voltage to the electrode module 155 and the electrode plate 160, the discharge solution may be converted into the discharge solution having hydroxide ions (OH⁻) by the electric field between the electrode module 155 and the electrode plate 160. Since the principle in which the discharge solution is ionized by the electric field force and is changed to the discharge solution state and then is sprayed is the same as the principle of the electro spraying that is commonly used, a more detailed description of the principle will be omitted.

As described above, the sterilization device 100 according to an embodiment of the present disclosure may atomize the base solution W accommodated in the housing 110 by using the atomization unit 117, may discharge the discharge solution through the discharge guide unit 120, and may spray the discharge solution as the discharge solution having hydroxide ions (OH⁻) by using the electric field formed by the electrode module 155 mounted in the discharge guide unit 120. As the discharge solution having the hydroxide ions (OH⁻) is sprayed into the air, humidification, sterilization, and deodorization functions may be performed.

Therefore, the sterilization device 100 according to an embodiment of the present disclosure is configured to spray the liquid-phase base solution W in the discharge solution state in which the discharge solution having hydroxide ions (OH⁻) by simply atomizing and ionizing the liquid-phase base solution W without using a separate deodorant or a separate air conditioner, so that not only additional contamination of the sterilization device 100 may be prevented but also irritating strong odors may not be generated and the sterilization device 100 is capable of being used in a clean, hygienic, and convenient manner.

In addition, in the sterilization device 100 according to an embodiment of the present disclosure, by preparing both the atomization unit 117 and the electrostatic spray unit 118, the atomized discharge solution may be generated by atomizing the liquid-phase base solution primarily, and the discharge solution may be converted into the discharge solution having the sterilization function using hydroxide ions (OH⁻). Therefore, the discharge solution may be more rapidly and effectively discharged than that of the conventional technology.

The electrostatic spray unit 118 according to another embodiment may include an ion generating electrode body having a first electrode and a second electrode that are facing each other with a glass plate that is a dielectric interposed therebetween, and may include a high-voltage alternating power connected by using the first electrode as a voltage applying electrode and by using the second electrode as a ground electrode.

In addition, a lighting apparatus 164 electrically connected to the voltage supply device 163 and configured to be operated by receiving power from the voltage supply device 163 may be further included. The lighting apparatus 164 is disposed such that the lighting apparatus 164 is configured to emit light into the inside of the housing 110 so that a soft light is generated around the housing 110, or the lighting apparatus 164 is disposed such that the lighting apparatus 164 is configured to emit light to the outside of the housing 110 so that a user can check whether an operation is performed or not.

The lighting apparatus 164 may be an LED light as an embodiment, and may be operated in connection with the operation of the sterilization device, a blower 220, or the electrostatic spray unit 118. Particularly, the lighting apparatus 164 is operated only at the time of operation of the sterilization device, the blower 220, or the electrostatic spray unit 118 so as to display an operation state.

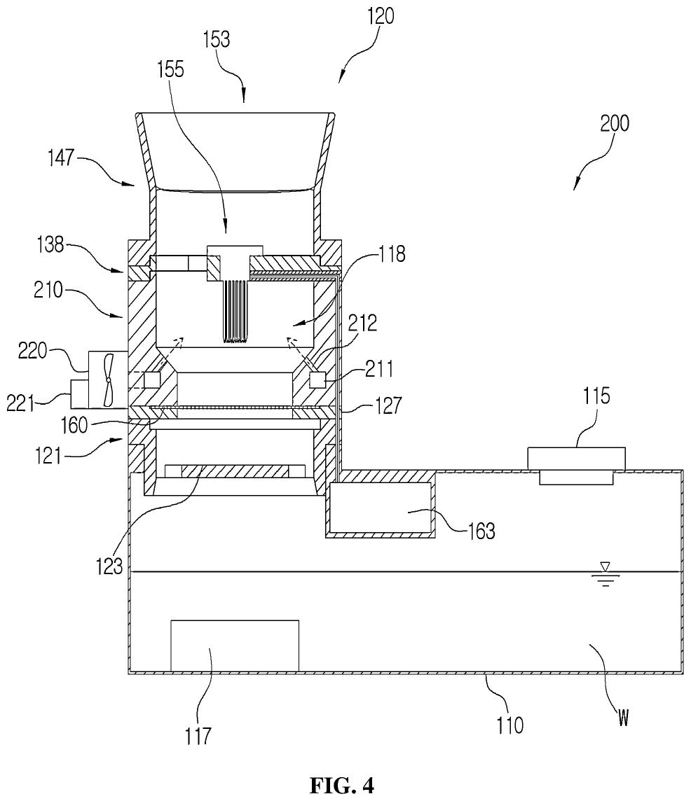

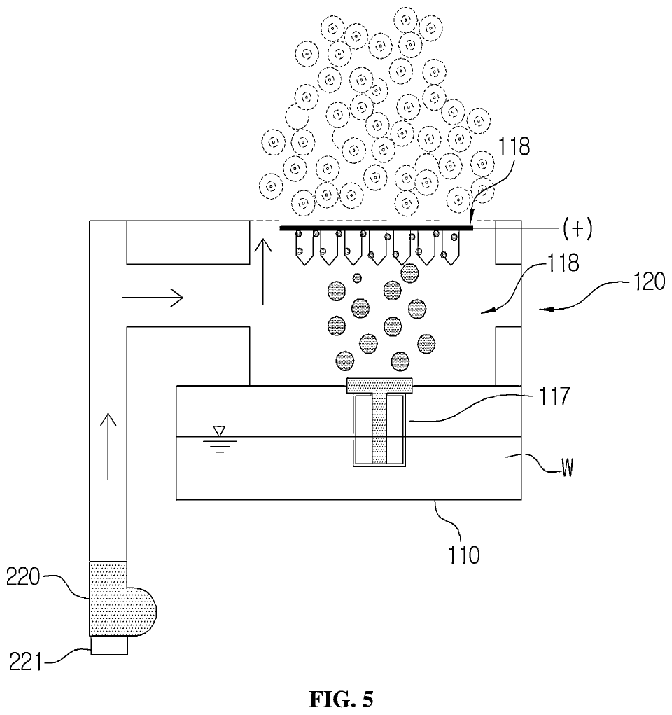

FIG. 4 is a cross-sectional view illustrating a modification example of the sterilization device according to an embodiment of the present disclosure, and FIG. 5 is a conceptual view illustrating a principle of operating the sterilization device illustrated in FIG. 4.

Referring to FIG. 4 and FIG. 5, a modification example of the discharge guide unit 120 of the sterilization device according to an embodiment of the present disclosure has most of configurations similar to those previously described, except that a structure of a guide tube part 210 is slightly modified.

An air flow path 211 through which air can pass is provided in an inner side of the guide tube part 210. In addition, a plurality of spraying ports 212 connected to the air flow path 211 is formed in the diffusion guide part 135 of the guide tube part 210. The blower 220 is mounted on a first side of the discharge guide unit 120 so as to blow air to the air flow path 211 of the guide tube part 210.

The blower 220 may be mounted such that a suction port 221 configured to suction air flowing thereinto is positioned on a side of the discharge guide unit 120. As an embodiment, the housing 110 may be disposed such that the housing 110 extends to a second side of the discharge guide unit 120, and the suction port 221 of the blower 220 may be mounted such that the suction port 221 of the blower 220 is positioned on a first side of the discharge guide unit 120. Accordingly, the blower 220 may suction fresh air with low humidity and low temperature by minimizing the influence of the liquid-phase base solution W or the voltage supply device 163 that are stored inside the housing 110.

In addition, the suction port 221 may be provided with a HEPA filter, so that air flowing from the outside into the blower 220 is capable of being purified primarily. Furthermore, an ultraviolet lighting device (not illustrated) configured to generate ultraviolet rays for sterilization may be further provided inside the blower 220, so that the air introduced from the suction port 221 is capable of being purified secondarily.

When the blower 220 is operated, the air that is blown from the blower 220 passes through the air flow path 211 and is sprayed into an internal space of the guide tube part 210 through the plurality of spraying ports 212. Since the plurality of spraying ports 212 is formed in the diffusion guide part 135 and is disposed obliquely toward the retainer 138, air passing through the plurality of spray ports 212 is capable of being sprayed obliquely toward the retainer 138. Therefore, after the discharge solution formed between the electrode module 155 and the electrode plate 160 is evenly distributed to the air inside the guide tube part 210, the discharge solution may be rapidly discharged to the outside through the discharge tube part 147.

The blower 220 may be provided with a power source common to the power source applied to the electrode module 155 and the electrode plate 160, or may be provided with a separate power source. When the blower 220 is provided with the common power source, the operation of the blower 220 may be controlled on/off by a separate controller (not illustrated) separately from the power application of the electrode module 155 and the electrode plate 160.

When a sterilization device 200 is operated, the liquid-phase base solution W accommodated in the housing 110 is atomized by the atomization unit 117 and is dispersed as an atomized discharge solution, and the atomized discharge solution passes through the discharge guide unit 120 and is converted into the discharge solution having hydroxide ions (OH⁻) by the electrostatic spray unit 118. In addition, as the blower 220 blows air into the discharge guide unit 120, the airflow containing the discharge solution is rapidly diffused to the air around the sterilization device 200, so that the surrounding air may be humidified, sterilized, and deodorized.

The principle of deodorizing contaminants by using the discharge solution having hydroxide ions (OH⁻) is as follows.

Ammonia: 2NH₃+6OH→N₂+6H₂O

Acetaldehyde: CH₃CHO+6OH+O₂→2CO₂+5H₂O

Acetic acid: CH₃COOH+4OH+O₂→2CO₂+4H₂O

Methane gas: CH₄₊₄OH+O₂→CO₂+4H₂O

Carbon monoxide: CO+2OH→CO₂+H₂O

Formaldehyde: HCHO+4OH→CO₂+3H₂O

In addition, the discharge solution having hydroxide ions (OH⁻) or hydroxide radicals (OH⁻) may perform a deodorization and sterilization function for various contaminants according to an oxidation reaction in which hydrogen atoms are extracted from bacterial cells and then the sterilization is performed.

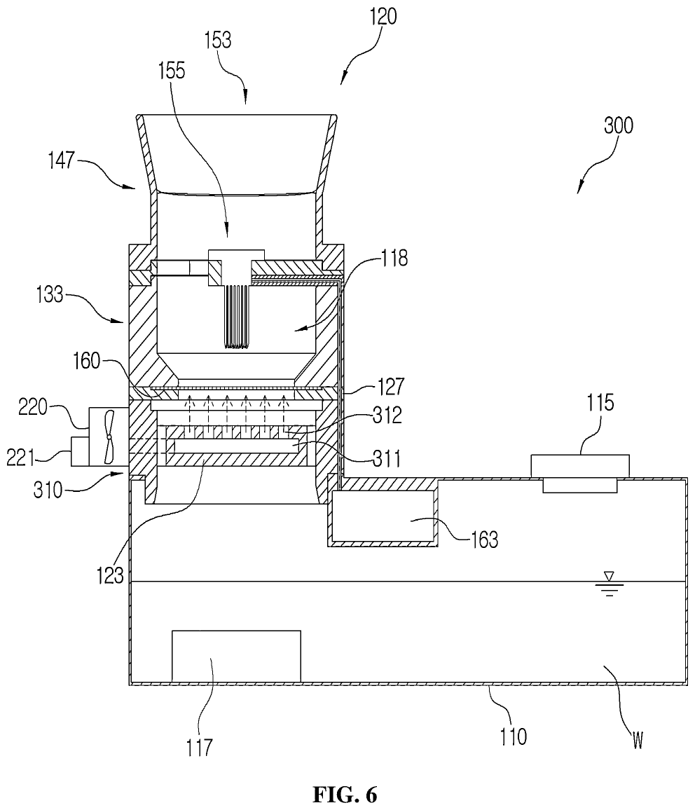

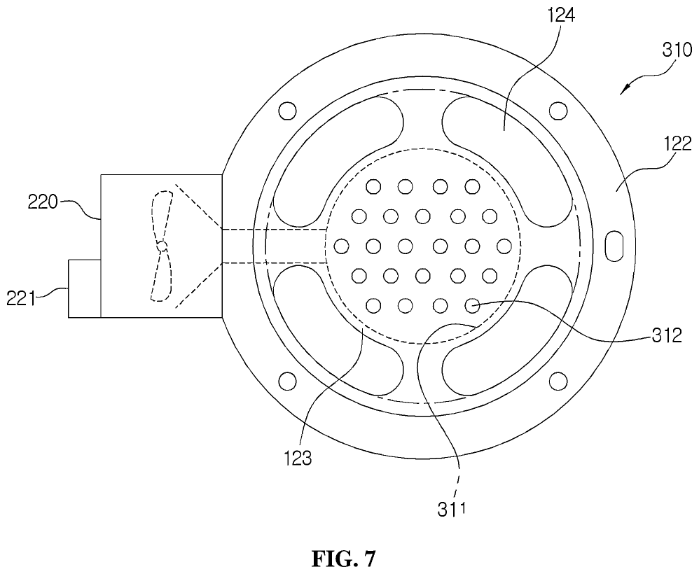

FIG. 6 is a cross-sectional view illustrating another modification example of the sterilization device according to an embodiment of the present disclosure, and FIG. 7 is a plan view illustrating a portion of the sterilization device illustrated in FIG. 6.

Referring to FIG. 6 and FIG. 7, another modification example of the discharge guide unit 120 of the sterilization device according to an embodiment of the present disclosure has most of configurations similar to those previously described, except that a structure of a dispersion tube part 310 is slightly modified.

An air flow path 311 through which air can pass is provided inside the dispersion tube part 310. In addition, a plurality of spraying ports 312 connected to the air flow path 311 is formed in the shielding part 123. The blower 220 is mounted on the first side of the discharge guide unit 120 so as to blow air to the air flow path 311 of the dispersion tube part 310.

When the blower 220 is operated, the air that is blown from the blower 220 passes through the air flow path 311 and is sprayed toward the electrode module 155 through the plurality of spraying ports 312. Therefore, the discharge solution formed at the inner side of the housing 110 by the atomization unit 117 and the discharge solution formed between the electrode module 155 and the electrode plate 160 may pass through the discharge guide unit 120 and may be evenly dispersed in the air, and then may be rapidly discharged to the outside through the discharge tube part 147.

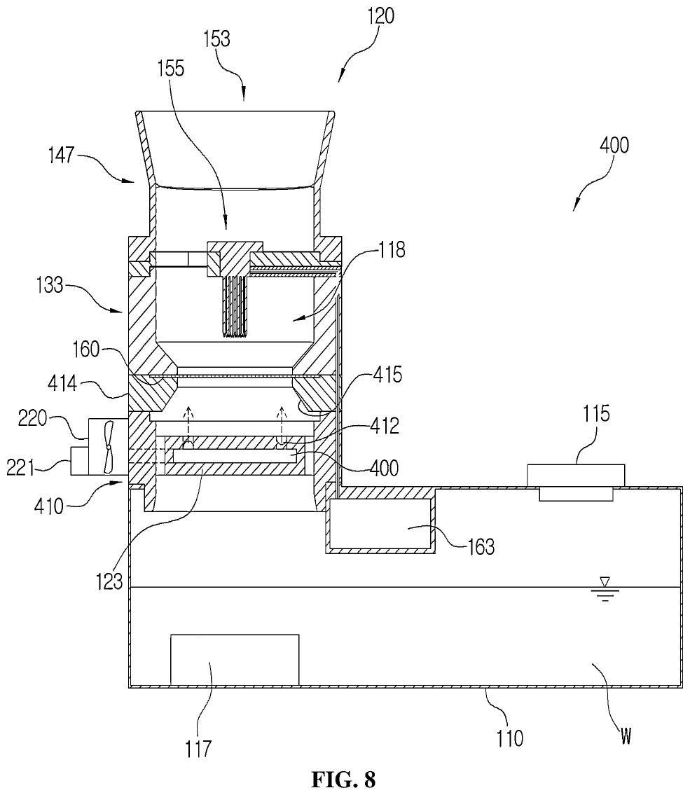

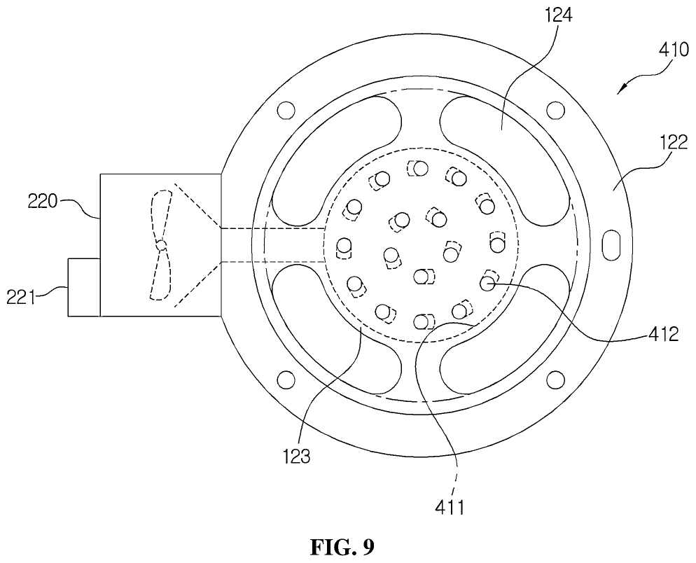

FIG. 8 is a cross-sectional view illustrating still another modification example of the sterilization device according to an embodiment of the present disclosure, and FIG. 9 is a plan view illustrating a portion of the sterilization device illustrated in FIG. 8.

Referring to FIG. 8 and FIG. 9, still another modification example of the discharge guide unit 120 of the sterilization device according to an embodiment of the present disclosure has most of configurations similar to those previously described, except that a structure of a dispersion tube part 410 and a structure of a support plate 414 are slightly modified.

An air flow path 411 through which air can pass is provided inside the dispersion tube part 410. In addition, a plurality of spraying ports 412 connected to the air flow path 411 is formed in the shielding part 123. The plurality of spraying ports 412 is disposed obliquely toward the electrode plate 160 so that air is capable of being sprayed in an inclined direction with respect to the electrode plate 160. Therefore, air passing through the plurality of spraying ports 412 may form a swirl flow.

The blower 220 is mounted on the first side of the discharge guide unit 120 so as to blow air to the air flow path 411 of the dispersion tube part 410.

The support plate 414 has the support plate passage 128 through which the discharge solution can pass. An inclination guide part 415 formed in a shape in which a width thereof is gradually decreased toward the electrode plate 160 is formed on a lower side of the support plate 414. The inclination guide part 415 may guide the swirl flow passing through the support plate passage 128 toward the center of the electrode plate 160.

When the blower 220 is operated, the air that is blown from the blower 220 passes through the air flow path 411 and is sprayed obliquely toward the electrode module 160 through the plurality of spraying ports 412. At this time, a swirl airflow containing the discharge solution is formed, and the swirl airflow containing the discharge solution is guided toward the center of the electrode plate 160 by the inclination guide part 415 of the support plate 414, and then the swirl airflow passes through the plurality of support plate passages 128 provided in the support plate 414. In addition, the discharge solution passing through the support plate 414 is converted into the discharge solution having hydroxide ions (OH⁻) from between the electrode module 155 and the electrode plate 160, and the swirl airflow containing the discharge solution may be rapidly discharged to the outside by sequentially passing through the guide tube part 133, the retainer 138, and the discharge tube part 147.

A sterilization device 400 according to still another embodiment of the present disclosure may generate and discharge a swirl airflow in the discharge guide unit 120 by blowing air through the blower 220 to the inside of the discharge guide unit 120, so that the discharge solution may be more evenly dispersed to the air and humidification, sterilization, and deodorization effects on the surrounding air may be increased.

Although the present disclosure has been described with exemplary examples, the scope of the present disclosure is not limited to the forms described and illustrated above.

For example, in the drawings, it is illustrated that the discharge guide unit 120 is assembled on the housing 110 such that the discharge guide unit 120 is capable of being attached to and detached from the housing 110, but the housing and the discharge guide unit may be manufactured as an integrated type.

In addition, in the drawings, it is illustrated that the blower 220 is mounted such that the blower 220 is configured to blow air into the inside of the discharge guide unit 120. However, the mounting position of the blower may be variously modified so that the blower is capable of blowing air into the inside of the housing or the inside of the discharge guide unit. In addition, the mounting number of the blowers may be variously modified.

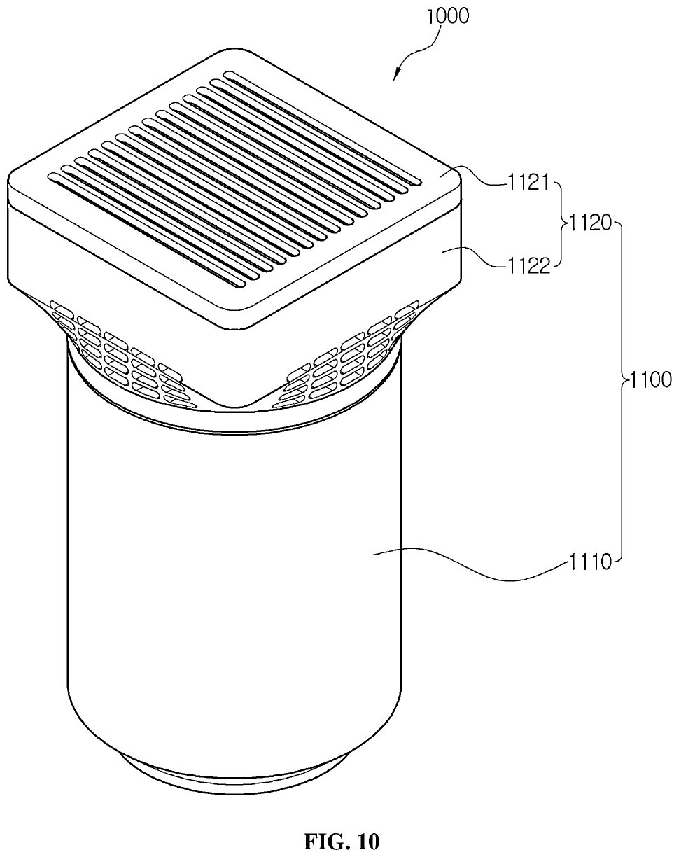

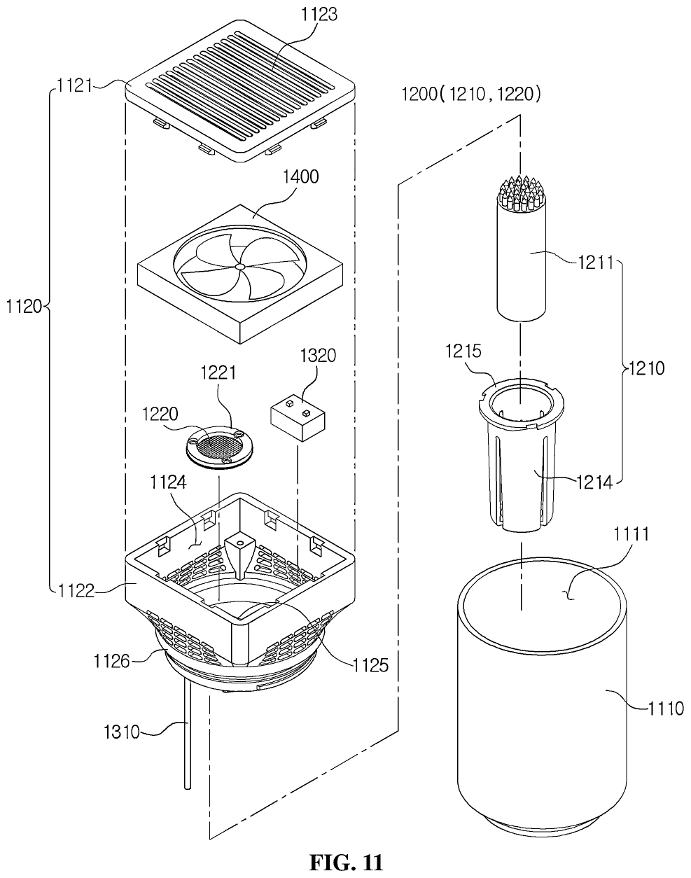

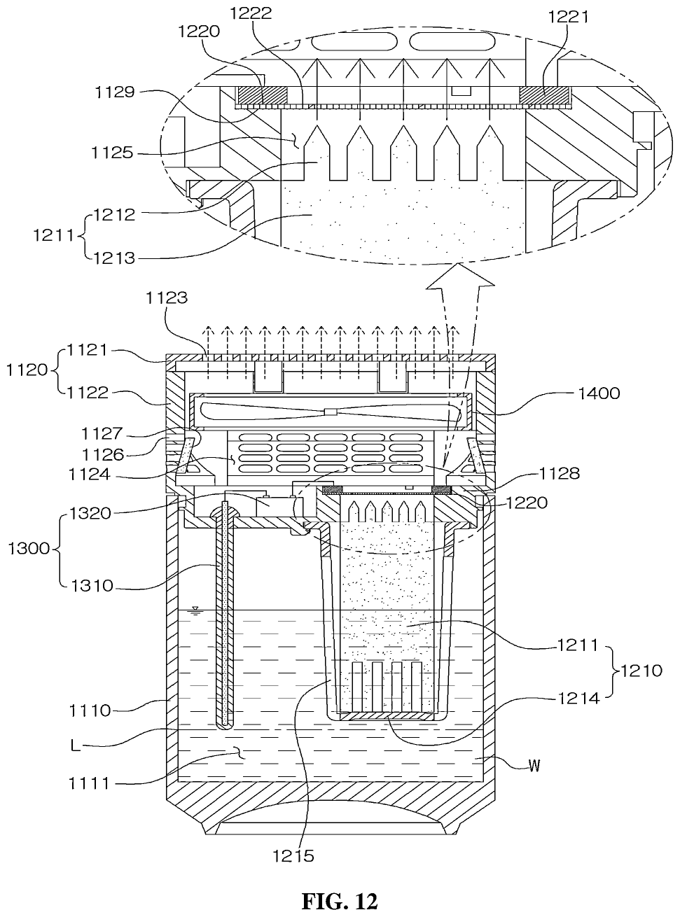

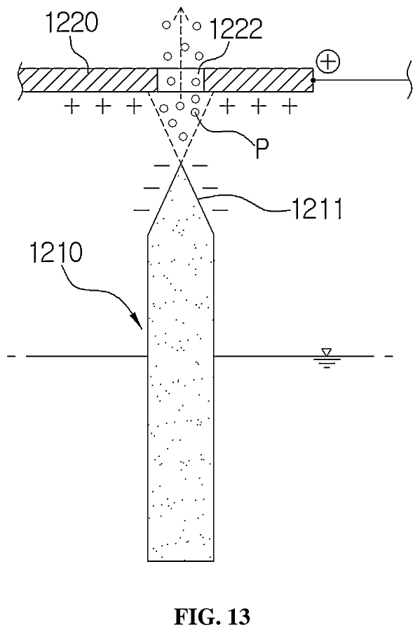

FIG. 10 is a perspective view illustrating the sterilization device according to another embodiment of the present disclosure, FIG. 11 is an exploded perspective view illustrating a detailed configuration of the sterilization device according to another embodiment of the present disclosure, FIG. 12 is a cross-sectional view illustrating an internal structure of the sterilization device according to another embodiment of the present disclosure, and FIG. 13 is a view illustrating a principle of spraying a discharge solution of the sterilization device according to another embodiment of the present disclosure.

Referring to FIG. 10 to FIG. 13, a sterilization device 1000 according to another embodiment of the present disclosure includes: a housing 1100 configured to store a liquid-phase base solution therein; an electrostatic spray unit 1200 configured to convert a discharge solution formed from the base solution stored in the housing 1100 into a discharge solution having a sterilization function using hydroxide ions (OH⁻); and a discharge guide unit (blowing chamber) 1124 coupled to a first side of the housing 1100, the discharge guide unit 1124 providing a discharge flow path 1123 configured to guide the discharge solution converted by the electrostatic spray unit 1200 to be discharged to the outside of the housing 1100. Furthermore, the electrostatic spray unit includes: a first electrode module (an electrode plate module) 1220 provided inside the housing and being provided with a plurality of discharge holes through which the discharge solution is discharged; and a second electrode module (a base solution absorption electrode module) 1210 disposed such that the second electrode module 1210 is spaced apart from the first electrode module 1220, the second electrode module 1210 being configured to transfer the liquid-phase base solution contained inside the housing to a position corresponding to the plurality of discharge holes of the first electrode module 1220, and the second electrode module 1210 being configured to convert the discharge solution by using a potential difference generated between the first electrode module 1220 and the second electrode module 1210.

First, the sterilization device 1000 according to another embodiment of the present disclosure is a device for sterilizing an indoor space by spraying micro-sized moisture droplets with sterilization functionality. Furthermore, the sterilization device 1000 serves to convert the liquid-phase base solution into the discharge solution having the sterilization function from inside the housing 1100 having an inner first side in which the liquid-phase base solution is stored, and serves to discharge solution to the outside of the housing 1100. Such a sterilization device includes the housing 1100, the electrostatic spray unit 1200, and a voltage application unit 1300.

Specifically, according to another embodiment of the present disclosure, the housing 1100 of the sterilization device 1000 includes a storage casing 1110 having an inner first side in which the liquid-phase base solution is stored, and includes an operation body 1120 coupled to the storage casing 1110. As will be described later, by having a structure in which the electrode plate module 1220 is separated and partitioned from a base solution storage chamber 1111 and the electrode plate module 1220 is stably coupled and fixed to an internal space of the operation body 1200, a separation distance between the electrode plate module 1220 and an absorption member tip part 1212 may be maintained constantly. In addition, through this, a stable electric field in an optimal state is formed between the absorption member tip part 1212 and the electrode plate module 1220, so that stable spraying of the discharge solution is capable of being realized. Accordingly, the sterilization function is capable of being performed stably and efficiently.

The storage casing 1110 is formed in a hollow cylindrical container shape with an open upper surface, and the base solution storage chamber 1111 capable of storing the base solution therein is formed inside the storage casing 1110.

The operation body 1120 is coupled to the open upper surface of the storage casing 1110 such that the operation body 1120 is capable of being attached to and detached from the open upper surface of the storage casing 1110, has an inner portion thereof provided with a blowing chamber 1124 separated and partitioned from the base solution storage chamber 1111, has an upper surface thereof provided with a blowing hole 1123, and has a lower surface thereof provided with a coupling hole 1125.

Such an operation body 1120 may be formed separately as a lower body 1122 formed in a hollow container shape with an open upper surface and an upper cover 1121 coupled to the open upper surface of the lower body 1122, and the blowing chamber 1124 is formed inside the lower body 1122. In a state in which the operation body 1120 is coupled to the storage casing 1110, the blowing chamber 1124 is separated and partitioned from the base solution storage chamber 1111 of the storage casing 1110 by a lower wall body of the lower body 1122. The coupling hole 1125 is formed through the lower wall body of the lower body 1122, and is configured such that the blowing chamber 1124 and the base solution storage chamber 1111 are in communication with each other. The blowing hole 1123 is formed vertically through the upper cover 1121.

In addition, a blowing fan 1400 configured to blow air toward the blowing hole 1123 is mounted in the blowing chamber 1124 of the operation body 1120, and an air inlet hole 1126 is formed in a side surface of the operation body 1120 so that external air is capable of entering into the blowing chamber 1124. When the blowing fan 1400 is operated, external air is introduced through the air inlet hole 1126, and an airflow discharged to the outside through the blowing hole 1123 is generated inside the blowing chamber 1124. According to such an airflow, a discharge solution P having the sterilization function described later is discharged to the outside together with the air through the blowing hole 1123.

At this time, since external foreign substances, bacteria, and so on may be introduced into the blowing chamber 1124 during the process in which external air is introduced into the blowing chamber 1124 through the air inlet hole 1126, a separate air filter 1127 may be mounted inside the side surface of the operation body 1120 such that external air introduced through the air inlet hole 1126 passes through air filter 1127. As an embodiment, a HEPA filter that filters out radioactive particles in air may be used as the air filter 1127.

As an embodiment, the air inlet hole 1126 may be formed on the entire side surface of the operation body 1120. As another embodiment, the air inlet hole 1126 may be formed only on a first side of the operation body 1120. Particularly, the air inlet hole 1126 may be formed in the operation body 1120 at a position as far as possible from the base solution absorption electrode module 1210 and the electrode plate module 1220, so that air is capable of being introduced through the air inlet hole 1126 positioned where the influence of the discharge solution formed by the base solution absorption electrode module 1210 and the electrode plate module 1220 is minimized.

Next, the electrostatic spray unit 1200 serves to convert the liquid-phase base solution stored inside the housing 1100 into the discharge solution having the sterilization function using hydroxide ions (OH⁻) from inside the housing 1100. Furthermore, the electrostatic spray unit 1200 includes the first electrode module and the second electrode module, and the liquid-phase base solution is converted into the discharge solution by the potential difference generated between the first electrode module and the second electrode module.

As illustrated in detail in FIG. 10 to FIG. 14, the first electrode module according to an embodiment of the present disclosure is the electrode plate module 1220 which is provided inside the housing 1100 and which is provided with a plurality of discharge holes 1222 through which the discharge solution is discharged.

In addition, as illustrated in detail in FIG. 10 to FIG. 14, the second electrode module according to an embodiment of the present disclosure is the base solution absorption electrode module 1210 disposed such that the base solution absorption electrode module 1210 is spaced apart from the first electrode module, the base solution absorption electrode module 1210 being configured to transfer the liquid-phase base solution stored inside the housing 1100 to a position corresponding to the plurality of discharge holes of the first electrode module, and the base solution absorption electrode module 1210 being configured to convert the liquid-phase base solution into the discharge solution by using the potential difference generated between the first electrode module and the base solution absorption electrode module 1210.

In the electrostatic spray unit 1200 according to an embodiment of the present disclosure having such a configuration, by a structure that will be described in detail later, the base solution absorbed in the base solution absorption electrode module 1210 is sprayed from the tip part 1212, the base solution being in a discharge solution state with the sterilization functionality by the electric field generated between the tip part and the electrode plate module 1220. Furthermore, the discharge solution passes through the discharge holes 1222.

That is, by absorbing the liquid-phase base solution into the absorption member 1211 and spraying the discharge solution from the tip part 1212 of the absorption member 1211 by the electric field, moisture is electrically ionized, and the discharge solution having hydroxide ions (OH⁻) is sprayed and discharged, thereby performing the sterilization function. Accordingly, the sterilization function is capable of being performed by simply using the liquid-phase base solution without using a separate deodorant or a separate air conditioner. Therefore, in addition to preventing additional contamination of the device, a cleaner, more hygienic, and convenient use of the device is capable of being realized since an irritating strong smell is not generated and a periodic operation by the user is not required.

The base solution absorption electrode module 1210 is coupled to the operation body 1120 such that the base solution absorption electrode module 1210 penetrates the coupling hole 1125 and protrudes downward, and has a lower end portion disposed in the base solution storage chamber 1111 of the storage casing 1111 such that the liquid-phase base solution W stored in the base solution storage chamber 1111 is absorbed into the base solution absorption electrode module 1210. The tip part 1212 is formed on an upper end portion of the base solution absorption electrode module 1210 such that the liquid-phase base solution absorbed through the lower end portion of the base solution absorption electrode module 1210 is capable of being sprayed upward.

The base solution absorption electrode module 1210 includes a support cover 1214 having a container shape with an open upper surface, the support cover 1214 having an upper end coupled to an outer peripheral portion of the coupling hole 1125 of the operation body 1120, and the support cover 1214 having a side surface provided with a base solution inlet opening part 1215 such that the liquid-phase base solution stored in the base solution storage chamber 1111 is capable of being introduced through the base solution inlet opening part 1215. Furthermore, the base solution absorption electrode module 1210 includes the absorption member 1211 seated and supported inside the support cover 1214 such that an upper end portion of the absorption member 1211 penetrates the coupling hole 1125, the absorption member 1211 being configured to absorb the liquid-phase base solution W introduced into an internal space of the support cover 1214. The absorption member 1211 may be formed in a shape in which the plurality of tip parts 1212 protrudes upward on an upper surface of a body part 1213, and may be configured such that the body part 1213 except the tip parts 1212 is disposed inside the support cover 1214 and the body part 1213 absorbs the liquid-phase base solution W.

Such an absorption member 1211 is formed of a material having an excellent absorption function for the liquid-phase base solution. For example, the absorption member 1211 may be formed of a porous material including polyethylene. Accordingly, as the liquid-phase base solution that is in contact with a lower portion of the absorption member 1211, the liquid-phase base solution is capable of being transferred to an upper portion of the absorption member 1211.

The electrode plate module 1220 may be formed of a metal that is an electrically conductive material, and is mounted inside the operation body 1120 such that the electrode plate module 1220 is spaced apart upward by a predetermined distance from the tip parts 1212 of the absorption member 1211. The discharge holes 1222 is formed in the electrode plate module 1220 such that the discharge solution P sprayed from the absorption member 1211 by the electric field is discharged.

At this time, as illustrated in FIG. 11 and FIG. 12, the absorption member 1211 is formed such that plurality of tip parts 1212 are provided, and the electrode plate module 1220 may have the plurality of discharge holes 1222 corresponding to the plurality of tip parts 1212. Furthermore, the plurality of discharge holes 1222 may be formed such that each of the plurality of discharge holes 1222 is positioned directly above each of the plurality of tip parts 1212. That is, the plurality of discharge holes 1222 may be formed in a position corresponding to the plurality of tip parts 1212, and may be disposed such that the plurality of discharge holes 1222 forms a concentric circle when viewed from above or below.

Meanwhile, the voltage application unit 1300 serves to apply a voltage between the base solution absorption electrode module 1210 and the electrode plate module 1220, and includes a needle electrode 1310 and a voltage supply device 1320.

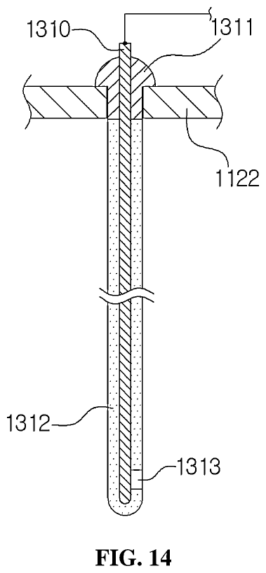

FIG. 14 is a view illustrating a shape of a needle electrode of the sterilization device according to another embodiment of the present disclosure.

Referring to FIG. 14, the needle electrode 1310 penetrates the operation body 1120 and is coupled to the operation body 1120 so as to protrude downward such that the needle electrode 1310 is submerged into and is in contact with the liquid-phase base solution W stored in the base solution storage chamber 1111. An upper end portion of the needle electrode 1310 is positioned inside the operation body 1120 and is connected to the voltage supply device 1320 via a wire, and a lower end portion of the needle electrode 1310 is positioned in the base solution storage chamber 1111 of the storage casing 1110 and is in contact with the liquid-phase base solution W. As an embodiment, the lower end portion of the needle electrode 1310 may extend to a position corresponding to the lower end portion of the base solution absorption electrode module 1210. Particularly, the lower end portion of the needle electrode 1310 and the lower end portion of the base solution absorption electrode module 1210 may extend to a lower operating limit L in which the base solution is required to be supplied to the base solution storage chamber 1111 and the sterilization device 1000 is not capable of being operated.

At this time, in order for the liquid-phase base solution W in the base solution storage chamber 1111 to not be introduced into the inside of the operation body 1120, a separate sealing member 1311 may be coupled to a region where the needle electrode 1310 penetrates the operation body 1120 and is coupled to the operation body 1120. The sealing member 1311 may surround the needle electrode 1310 and may cover a penetration hole of the operation body 1120 through which the needle electrode 1310 penetrates. As an embodiment, the sealing member 1311 may be formed of an elastic material such as rubber.

The voltage supply device 1320 is disposed inside the operation body 1120 and is configured to supply a voltage to the electrode plate module 1220 and the needle electrode 1310 so that a potential difference occurs between the liquid-phase base solution W in the base solution storage chamber 1111 and the electrode plate module 1220. As an embodiment, the voltage supply device 1320 may be a charging device such as a battery or a storage battery that supplies power by using a charged electrical energy, or may be a power conversion device connected to a separate domestic or industrial power supply device (for example, an electrical outlet) and configured to supply electrical energy.

The electrode plate module 1220 and the needle electrode 1310 are connected to the voltage supply device 1320 through respective wires, and the needle electrode 1310 is in contact with the liquid-phase base solution W in the base solution storage chamber 1111, so that the voltage supplied from the voltage supply device 1320 is applied to the liquid-phase base solution W in the base solution storage chamber 1111 through the needle electrode 1310.

The electrode plate module 1220 is connected to the voltage supply device 1320, and a high-voltage is applied to the electrode plate module 1220 through the voltage supply device 1320. Furthermore, the needle electrode 1310 may be configured such that a ground power source is connected to the needle electrode 1310. Through this, the ground power source is applied to liquid-phase base solution W in the base solution storage chamber 1111.

On the contrary, the needle electrode 1310 may be connected to the voltage supply device 1320, and a high-voltage may be applied to the needle electrode 1310 through the voltage supply device 1320. Accordingly, the high-voltage may be applied to the liquid-phase base solution in the base solution storage chamber 1111, and the electrode plate module 1220 may be configured such that a ground power source is connected to the electrode plate module 1220.

When a potential difference occurs between the liquid-phase base solution W in the base solution storage chamber 1111 and the electrode plate module 1220 by such a voltage supply device 1320, an electric field by the potential difference is generated in the space between the tip part 1212 of the absorption member 1211 in which the liquid-phase base solution W in the base solution storage chamber 1111 is absorbed and the electrode plate module 1220.

The liquid-phase base solution absorbed in the absorption member 1211 is converted into the discharge solution P state by the electric field generated between the tip part 1212 of the absorption member 1211 and the electrode plate module 1220, and is sprayed toward the electrode plate module 1220. The discharge solution P sprayed toward the electrode plate module 1220 passes through the discharge hole 1222 of the electrode plate module 1220 and is introduced into the blowing chamber 1124 of the operation body 1120. Furthermore, the discharge solution P that is introduced into the blowing chamber 1124 is discharged to the outside through the blowing hole 1123 according to the flow of air in the blowing chamber 1124.

For example, the liquid-phase base solution W in the base solution storage chamber 1111 is absorbed in the absorption member 1211 and is moved toward the tip part 1212 by a capillary phenomenon. In this state, as illustrated in FIG. 13, when a high-voltage is applied to the electrode plate module 1220 and a ground power is applied to the liquid-phase base solution W in the base solution storage chamber 1111 through the needle electrode 1310, the electric field force generated between the liquid-phase base solution W absorbed in the absorption member 1211 and the electrode plate module 1220 is concentrated on the tip part 1212 of the absorption member 1211, so that the moisture at the tip part 1212 of the absorption member 1211 is converted into the discharge solution P state by the concentrated electric field force and the discharge solution P is sprayed toward the electrode plate module 1220 at a high speed. The discharged solution P sprayed as described above passes through the discharge hole 1222 of the electrode plate module 1220 and is introduced into the blowing chamber 1124, and is discharged to the outside through the blowing hole 1123 according to the flow of air in the blowing chamber 1124 by the blowing fan 1400.

The size of the discharge solution P sprayed from the tip part 1212 of the absorption member 1211 may be modified according to the size of the electric field. By appropriately adjusting the size of the potential difference by the voltage supply device 1320, the size of the discharge solution P may be formed at the nanoparticle level.

Since the principle in which moisture at the tip part 1212 of the absorption member 1211 is converted into the discharge solution P state by the electric field and is sprayed toward the electrode plate module 1220 is the principle in the same manner as the principle of an electro spraying that is commonly used, further detailed descriptions of the principle of spraying the discharge solution P will be omitted.

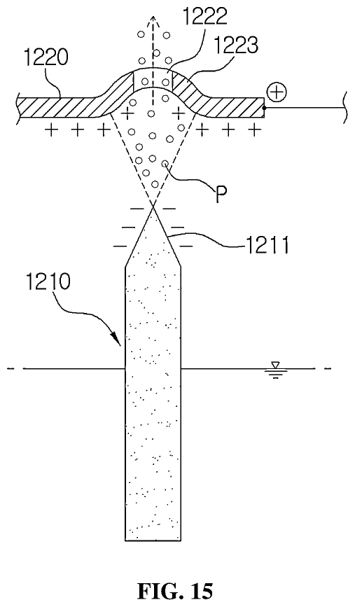

FIG. 15 is a view illustrating a principle of spraying a discharge solution according to a modification example of the sterilization device according to another embodiment of the present disclosure.

Referring to FIG. 15, the electrode plate module 1220 is disposed such that the electrode plate module 1220 is spaced apart from the upper portion of the tip part 1212 of the absorption member 1211 as described above. In the electrode module 1220, as illustrated in FIG. 14, an embossed part 1223 that protrudes upward in a curved manner may be formed at a point vertically above the tip part 1212, and the discharge hole 1222 may be formed at the center portion of the embossed part 1223.

As such, when the embossed part 1223 is formed in the electrode plate module 1220 such that the embossed part 1223 is curved in a concave shape toward the tip part 1212, an opposing region of the electrode plate module 1220 that influences the electric field at the tip part 1212 of the absorption member 1211 is relatively increased compared to that of a flat plate configuration, so that the force of the electric field concentrated on the tip part 1212 is further increased. Therefore, the spray amount of the discharge solution P sprayed from the tip part 1212 is increased. Furthermore, as the force of the electric field is more concentrated, the particle size of the discharge solution P may be formed more finely.

Since the size of the discharge solution P sprayed in this manner is very fine at the nano-level, and is not affected by the weight thereof, the suspension time is increased and, accordingly, the falling amount of the discharge solution P due to the weight thereof is significantly reduced, so that the external discharge amount of the discharge solution P is increased to the maximum without waste.

Particularly, since moisture in the discharge solution P is ionized by the electric field, hydroxide ions (OH⁻) are distributed on the surface of the discharge solution P. Since such hydroxide ions perform the sterilization function, the discharge solution P generated according to an embodiment of the present disclosure has the sterilization function. Therefore, when the hydroxide ions are discharged to the indoor space through the blowing hole 1123, the sterilization function is performed for the corresponding indoor space.

According to such a structure, in the sterilization device according to an embodiment of the present disclosure, the liquid-phase base solution W stored in the internal space of the storage casing 1110 is absorbed into the absorption member 1211, and the discharge solution Pis sprayed from the tip part 1212 of the absorption member 1211 by the electric field, so that the discharge solution P having hydroxide ions is sprayed by the electric field, thereby performing the sterilization function. Therefore, without using a separate deodorant or an air conditioner described in the background art, the liquid-phase base solution is absorbed and sprayed simply into an ionized discharge solution state, and additional contamination of the device is prevented and an irritating strong smell is not generated, and periodic operation by the user is not required, so that cleaner, hygienic, and convenient use is capable of being realized.

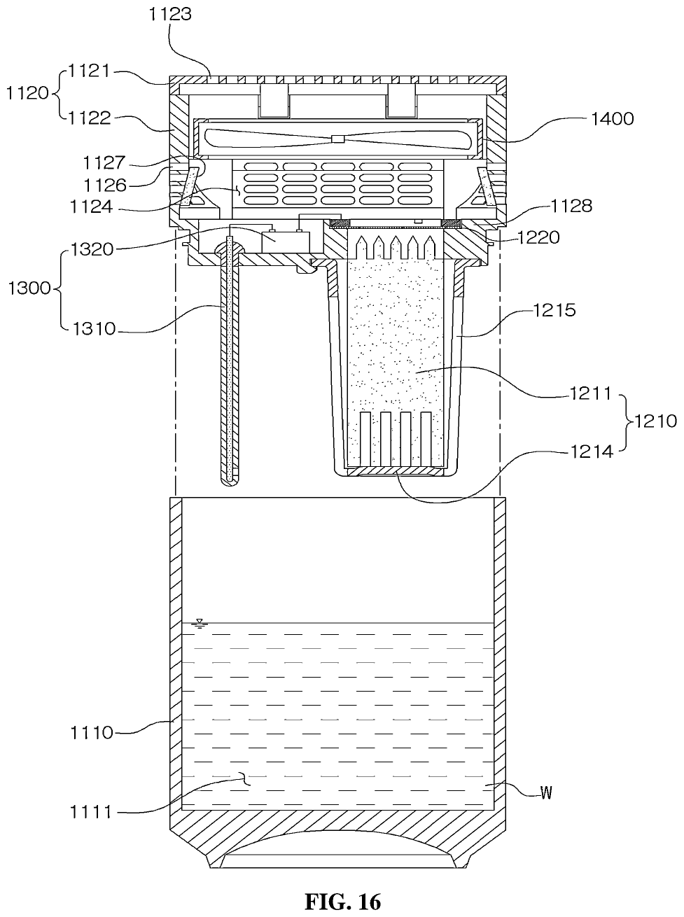

FIG. 16 is a view illustrating a state in which an operation body of the sterilization device according to another embodiment of the present disclosure is separated.

In the process of using such a sterilization device referring to FIG. 16, the operation body 1120 is separated from the storage casing 1110 first as illustrated in FIG. 16. When the operation body 1120 is separated, all components such as the base solution absorption electrode module 1210, the electrode plate module 1220, the needle electrode 1310, and the voltage supply device 1320 are separated from the storage casing 1110 while the components are coupled to the operation body 1120. In this state, the liquid-phase base solution W is input into the base solution storage chamber 1111 of the storage casing 1110. After the liquid-phase base solution is input, the operation body 1120 is coupled to the storage casing 1110 again. In this state, the liquid-phase base solution in the base solution storage chamber 1111 is absorbed into the absorption member 1211, and the needle electrode 1310 is in contact with the liquid-phase base solution in the base solution storage chamber 1111. Then, the voltage supply device 1320 and the blowing fan 1400 may be operated by operating a separate operation switch (not illustrated). According to such operation, the discharge solution P is sprayed from the tip part 1212 of the absorption member 1211 and is discharged to the outside through the blowing hole 1123.

When the storage amount of the liquid-phase base solution W stored in the base solution storage chamber 1111 is decreased after a specific time of use, the liquid-phase base solution W may be replenished while the operation body 1120 is separated, and the liquid-phase base solution W may be used. Particularly, as described above, in a state in which the operation body 1120 is coupled to the base solution storage chamber 1111, the lower operating limit L may be formed at the position of the lower end portion of the needle electrode 1310 or the position of the lower end portion of the base solution storage chamber 1210. Furthermore, when the storage amount of the liquid-phase base solution W stored in the base solution storage chamber 1111 is equal to or less than the lower operating limit L, the operation of the sterilization device 1000 is stopped by a separate operation stopping mechanism, and a signal (warning light, a button, warning sound, or the like) that requires the liquid-phase base solution water to be supplied in the base solution storage chamber 1111 may be generated.

In this situation, when the operation body 1120 is separated from the storage casing 1110, the needle electrode 1310 that protrudes downward from the operation body 1120 is exposed to the outside. When the needle electrode 1310 is exposed to the outside, the user may contact the needle electrode 1310, and an electric shock may occur in this situation.

In order to prevent such a risk of the occurrence of the electric shock, a separate insulation material 1312 may be coupled to the portion of the needle electrode 1310 that protrudes downward from the operation body 1120 such that the separate insulation material 1312 surrounds an outer circumferential surface of the needle electrode 1310, and an exposure hole 1313 may be formed on a lower end portion of the insulation material 1312 so that the needle electrode 1310 is exposed to the outside.

As an embodiment, the exposure hole 1313 may be formed on a side surface of the insulation material 1312 as illustrated in the drawings, and the lower operating limit L may be positioned at a lower end of the exposure hole 1313.

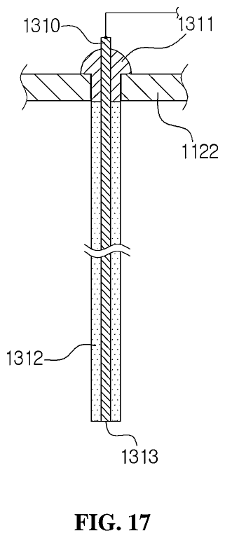

FIG. 17 is a view illustrating a modification example of the shape of the needle electrode of the sterilization device according to another embodiment of the present disclosure.

Referring to FIG. 17, as another embodiment, the exposure hole 1313 may be formed on a lower surface of the insulation material 1312 and, accordingly, the lower operating limit L may be positioned at the lowest end of the needle electrode 1310. Accordingly, the risk of the occurrence of the electric shock is reduced by minimizing contact with the user when the needle electrode 1310 is exposed to the outside. Furthermore, by lowering the position of the lower operating limit L, there is an effect that the liquid-phase base solution W stored in the storage casing 1110 may be utilized to the maximum extent.

According to this configuration, the outer circumferential surface of the needle electrode 1310 is protected by the insulation material 1312, so that the user is prevented from being in contact with the needle electrode 1310. Furthermore, in a state in which the needle electrode 1310 is submerged in the liquid-phase base solution W in the base solution storage chamber 1111, the needle electrode 1310 is capable of being in contact with the liquid-phase base solution W by being in communication with the liquid-phase base solution W through the exposure hole 1313.

Meanwhile, although not illustrated, a separate operation stopping mechanism for stopping the operation state of the voltage supply device 1320 when the operation body 1120 is separated from the storage casing 1110 may be provided. In this situation, power is not applied to the user even when the user is in contact with the needle electrode 1310, so that the risk of the occurrence of the electric shock may be prevented. The operation stopping mechanism for the voltage supply device 1320 may be configured as a switch-type operation stopping mechanism configured to be physically pressed by the operation body 1120 in the process of coupling the operation body 1120 to the storage casing 1110 and configured to be released when the operation body 1120 is separated from the storage casing 1110. Furthermore, the operation stopping mechanism may be modified in various manners such as a controlled-type operation stopping mechanism which is provided with a detection sensor configured to detect whether the operation body 1120 is coupled to the storage casing 1110 or not and which is configured such that an operation state of the voltage supply device 1320 is controlled according to a detection value of the detection sensor.

Meanwhile, in a state in which the operation body 1120 is coupled to the storage casing 1110, the blowing chamber 1124 of the operation body 1120 and the base solution storage chamber 1111 of the storage casing 1110 are separated and partitioned from each other and are in communication with each other through the coupling hole 1125. As such, as the blowing chamber 1124 of the operation body 1120 is separated and partitioned from the base solution storage chamber 1111 of the storage casing 1110, various electronic components such as the electrode plate module 1220, the voltage supply device 1320, and so on mounted inside the operation body 1120 may be protected from the liquid-phase base solution.

The absorption member 1211 is disposed below the coupling hole 1125, and the tip part 1212 of the absorption member 1211 is disposed such that the tip part 1212 passes through the coupling hole 1125. The electrode plate module 1220 is disposed in the internal space of the operation body 1120 such that the electrode plate module 1220 is spaced apart upwardly from the tip part 1212. At this time, an electrode plate module support part 1128 that protrudes upward along the outer circumference of the coupling hole 1125 is formed on an inner lower bottom surface of the operation body 1120, and a step part 1129 is formed on an upper end of the electrode plate module support part 1128. The electrode plate module 1220 is seated on and coupled to the step part 1129 of the electrode plate module support part 1128. In this state, a separate fixing ring 1221 is fixed to the step part 1129 along a circumference of the upper surface of the electrode plate module 1220, thereby pressing and fixing the electrode plate module 1220.

According to this configuration, the electrode plate module 1220 may be separated and partitioned from the base solution storage chamber 1111, and may be stably coupled and fixed to the internal space of the operation body 1120. In addition, the separation distance between the electrode plate module 1220 and the tip part 1212 of the absorption member 1211 may be maintained constantly. Through this, a stable electric field in an optimal state is generated between the electrode plate module 1220 and the tip part 1211, so that stable spraying of the discharge solution P is capable of being realized.

Meanwhile, although not illustrated, a spacer insertion groove (not illustrated) for inserting and fixing a separate spacer (not illustrated) may be formed in a bottom surface or a side surface of the step part 1129, and the position of the electrode plate module 1220 may be moved upward by inserting the spacer into the spacer insertion groove as required. Accordingly, the intensity of the electric field generated between the electrode plate module 1220 and the tip part 1212 may be modified, thereby being capable of adjusting the spraying amount of the discharge solution P. When the separation distance between the electrode plate module 1220 and the tip part 1212 is reduced, the spraying amount of the discharge solution Pis increased as the intensity of the electric field is increased. When the separation distance between the electrode plate module 1220 and the tip part 1212 is increased, the spraying amount of the discharge solution P is decreased as the intensity of the electric field is decreased.

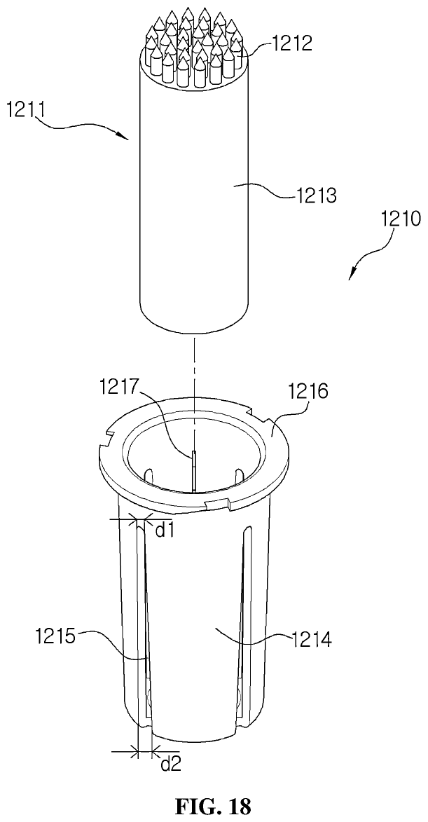

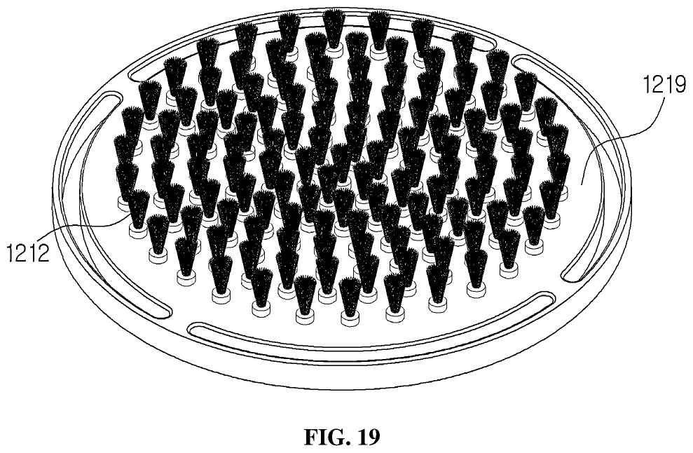

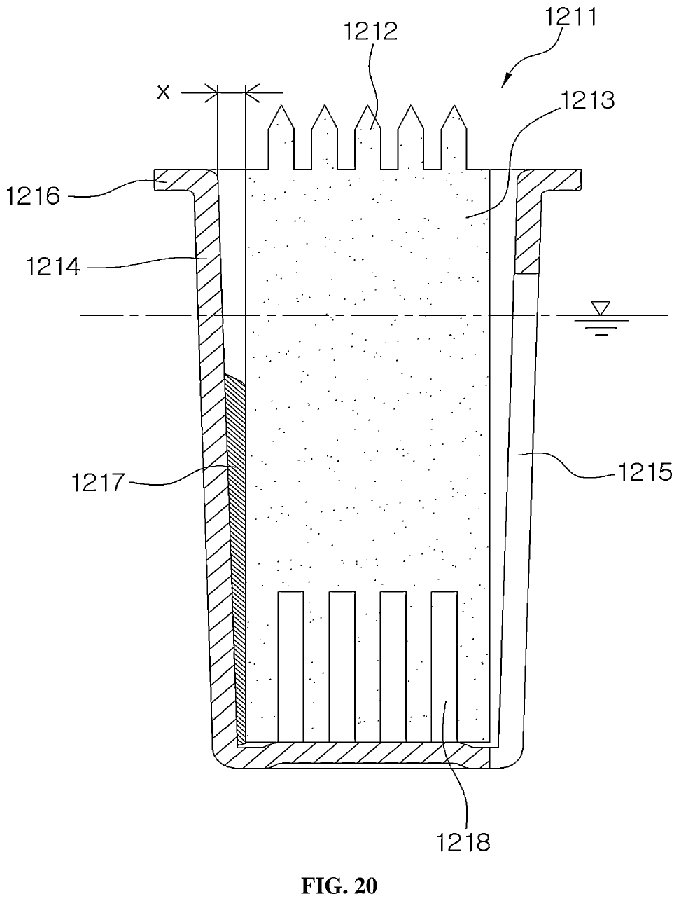

FIG. 18 is an exploded perspective view illustrating a configuration of a base solution absorption electrode module of the sterilization device according to another embodiment of the present disclosure, FIG. 19 is a perspective view illustrating a tip part of the sterilization device according to another embodiment of the present disclosure, and FIG. 20 is a cross-sectional view illustrating a coupling structure of the base solution absorption electrode module of the sterilization device according to another embodiment of the present disclosure.

Referring to FIG. 18 to FIG. 20, the base solution absorption electrode module 1210 includes the support cover 1214 and the absorption member 1211 as described above. Furthermore, the support cover 1214 is formed in the hollow cylindrical shape with the open upper surface, and a flange part 1216 is formed on a circumference of an upper end of the support cover 1214. The support cover 1214 is coupled to the lower surface of the operation body 1120 through the flange part 1216 such that the support cover 1214 is capable of being attached to and detached from the operation body 1120, and a catching hook capable of being engaged to the flange part 1216 may be formed on the lower surface of the operation body 1120. Through the detachable structure of the support cover 1214, the absorption member 1211 is capable of being replaced by separating the support cover 1214 from the operation body 1120.

The support cover 1214 is formed such that the flange part 1216 is coupled to the operation body 1120 along the outer circumference of the coupling hole 1125 of the operation body 1120, and the base solution inlet opening part 1215 is formed in the side surface of the support cover 1214 such that the liquid-phase base solution W stored in the base solution storage chamber 1111 is introduced through the base solution inlet opening part 1215.

The absorption member 1211 is seated and supported inside the support cover 1214 such that the upper end portion of the absorption member 1211 penetrates the coupling hole 1125, and absorbs the liquid-phase base solution W introduced through the base solution inlet opening part 1215. Such an absorption member 1211 is formed in the shape in which the plurality of the tip parts 1212 protrudes upward on the upper surface of the body part 1213, the body part 1213 except the tip parts 1212 is disposed inside the support cover 1214, and the tip parts 1212 are disposed such that the tip parts 1212 penetrate the coupling hole 1125.

As such, when the absorption member 1211 is formed such that the absorption member 1211 has a single body part 1213 and the plurality of tip parts 1212, a larger amount of the liquid-phase base solution is capable of being absorbed through the body part 1213 having the relatively large size, so that the spraying amount of the discharge solution P sprayed from the tip parts 1212 may be increased. In addition, since the body part 1213 has the relatively large size, the absorption storage amount of the liquid-phase base solution through the body part 1213 is increased, the liquid-phase base solution is smoothly absorbed and transferred to the plurality of tip parts 1212, so that the discharge solution P may be stably discharged from the plurality of tip parts 1212.

At this time, as illustrated in FIG. 20, a plurality of concave grooves 1218 may be formed in the body part 1213. By the plurality of concave grooves 1218, the contact area with the liquid-phase base solution W in the base solution storage chamber 1111 is increased, so that more base solution may be absorbed in a shorter period of time. Accordingly, when the sterilization device according to an embodiment of the present disclosure is initially operated, the liquid-phase base solution may be absorbed and then may be sprayed and discharged in a shorter period of time.

As illustrated in FIG. 19, in an embodiment, the each of the tip parts 1212 may be formed of a carbon fiber bundle formed of a plurality of carbon fibers. That is, the tip part 1212 is formed of the carbon fiber bundle in which the plurality of carbon fibers is bound as a bundle, and each of the carbon fiber bundles may be inserted into and fixed to a bundle support stand 1219 in which a plurality of bundle coupling holes is formed.

Each of the carbon fiber bundles may be inserted into each of the bundle coupling holes while being in a state in which each of the carbon fiber bundles is bound as a bundle, and is capable of being conveniently withdrawn and replaced.

The plurality of carbon fibers is bound as the carbon fiber bundle while being in contact with each other. However, when a voltage is applied to the plurality of carbon fibers, the plurality of carbon fibers is charged to the same polarity, so that a repulsive force may be generated between end portions of the plurality of carbon fibers. Accordingly, when a voltage is applied, the plurality of carbon fibers bound as a bundle is splayed in a side direction since the repulsive force in which the end portion of each of the plurality of carbon fibers is spaced apart from each other is generated, so that the discharge solution P may be sprayed in a scattered state.

The bundle support stand 1219 may be a portion of the support cover 1214, or may be integrally coupled to the support cover 1214 and may fix the carbon fiber bundles that are inserted into and fixed to the bundle coupling holes. The bundle support 1219 may be positioned such that the bundle support 1219 is in direct contact with the body part 1213, or may be configured to position the carbon fiber bundles such that each of the carbon fiber bundles is in contact with the body part 1213.

As another embodiment, the tip part 1212 may be a fiber bundle in which a plurality of unit fibers is coupled to each other.

The unit fiber may be selected from a group consisting of glass fiber, carbon fiber, metal fiber, aramid fiber, polyamide fiber, and a mixture or a combination thereof.

The unit fiber may be selected from a group consisting of a unit fiber having a circular cross-sectional area and a unit fiber having a non-circular cross-sectional area, and may preferably exist in the form of a short-fiber. Particularly, the unit fiber may preferably exist in the form of a stable fiber with a length ranging from 0.1 mm to 50 mm or in the form of a continuous fiber (roving).

Meanwhile, the base solution inlet opening part 1215 is formed on the side surface of the support cover 1214 so that the liquid-phase base solution Wis introduced through the base solution inlet opening part 1215 and is absorbed into the absorption member 1211. At this time, when an inner surface of the absorption member 1211 and an inner surface of the support cover 1214 are in contact with each other, the absorption member 1211 is in contact with the liquid-phase base solution W only through an opening area of the base solution inlet opening part 1215, so that the absorption member 1211 is not in contact with the liquid-phase base solution through the entire area of the absorption member 1211 and the contact area of the absorption member 1211 with the liquid-phase base solution may be relatively reduced.

In order to increase the contact area of the absorption member 1211 with the liquid-phase base solution, a plurality of protrusion ribs 1217 may be formed on an inner surface of the support cover 1214 such that a separation distance X is maintained between the inner surface of the support cover 1214 and an outer surface of the absorption member 1211 and the plurality of protrusion ribs 1217 presses and supports the outer surface of the absorption member 1211.

As an embodiment, the protrusion rib 1217 may protrude inwardly from the support cover 1214. Particularly, the protrusion rib 1217 may be positioned such that the protrusion rib 1217 and the base solution inlet opening part 1215 are disposed in a staggered manner. Specifically, the plurality of protrusion ribs 1217 may be disposed between the plurality of base solution inlet opening parts 1215.

Through this, the absorption member 1211 is maintained in the support cover 1214 in a state in which the absorption member 1211 is spaced apart from the inner circumferential surface of the support cover 1214 by the separation distance X. Therefore, the liquid-phase base solution W in the base solution storage chamber 1111 is introduced into the internal space of the support cover 1214, i.e., the separation space between the absorption member 1211 and the inner circumferential surface of the support cover 1214. Therefore, the contact area of the absorption member 1211 with the liquid-phase base solution is relatively increased, so that the absorption storage amount of the liquid-phase base solution is increased.

As illustrated in FIG. 20, in an embodiment, the support cover 320 may have a shape in which the outer diameter and the inner diameter of the support cover 1214 are reduced toward the lower side of the support cover 1214, and the protrusion rib 1217 may have a shape in which a height of the protrusion rib 1217 is reduced toward the lower side of the protrusion rib 1217. Accordingly, the inner diameter of the support cover 1214 by the protrusion rib 1217 is maintained constant, and the area in contact with the absorption member 1211 is increased, so that the absorption member 1211 may be solidly fixed.

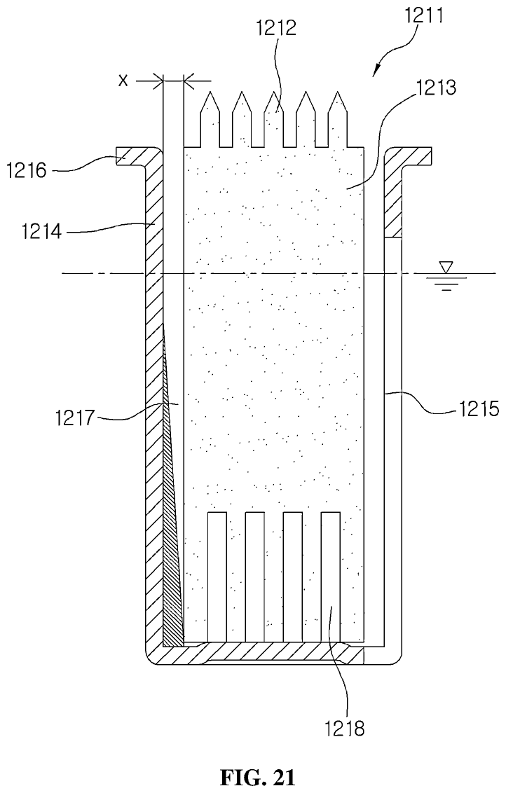

FIG. 21 is a cross-sectional view illustrating a modification example of the coupling structure of the base solution absorption electrode module of the sterilization device according to another embodiment of the present disclosure.

Referring to FIG. 21, as another embodiment, the support cover 1214 may be a cylindrical shape in which the outer diameter and the inner diameter of the support cover 1214 are constantly maintained. On the other hand, since the height of the protrusion rib 1217 is gradually increased toward the lower side of the protrusion rib 1217, the inner diameter of the support cover 1214 by the protrusion rib 1217 may be gradually reduced as the inner diameter of the protrusion rib 1217 moves downward. That is, the protrusion rib 1217 is in contact with the outer surface of the absorption member 1211 only at the lower end portion of the absorption member 1211. Accordingly, the absorption member 1211 is conveniently inserted into the inside of the support cover 1214, and there is an effect that the absorption member 1211 has an increased absorption speed and an increased storage amount of the liquid-phase base solution.

As still another embodiment, the support cover 1214 may have a shape in which the outer diameter and the inner diameter of the support cover 1214 are reduced toward the lower side of the support cover 1214, and the protrusion rib 1217 may have a shape in which the height of the protrusion rib 1217 is maintained constant.

Meanwhile, as described above, when the operation body 1120 is separated and withdrawn from the storage casing 1110, the support cover 1214 and the absorption member 1211 are also withdrawn from the storage casing 1110 in a state in which the support cover 1214 and the absorption member 1211 are coupled to the operation body 1120. At this time, the introduced liquid-phase base solution may remain in the internal space of the support cover 1214. When the liquid-phase base solution remains, the liquid-phase base solution may flow to the outside through the base solution inlet opening part 1215, so that the surrounding environment may be contaminated.

In order to prevent this situation, the base solution inlet opening part 1215 of the support cover 1214 may be formed in a shape in which the lower end of the base solution inlet opening part 1215 is open from the side surface of the support cover 1214 and the opening width of the base solution inlet opening part 1215 is increased toward the lower side of the base solution inlet opening part 1215.

That is, as illustrated in FIG. 18, the base solution inlet opening part 1215 may be formed such that an upper opening width d1 is smaller than a lower opening width d2. As such, the lower end of the base solution inlet opening part 1215 is open, and the lower opening width d2 is formed larger than the upper opening width d1, so that the liquid-phase base solution inside the support cover 1214 may be rapidly discharged through the base solution inlet opening part 1215 during the process in which the support cover 1214 is withdrawn from the storage casing 1110. Therefore, in a state in which the support cover 1214 is withdrawn, the liquid-phase base solution is not left inside the support cover 1214 or is minimized, so that the liquid-phase base solution is prevented from flowing to the outside and contaminating the surrounding environment.

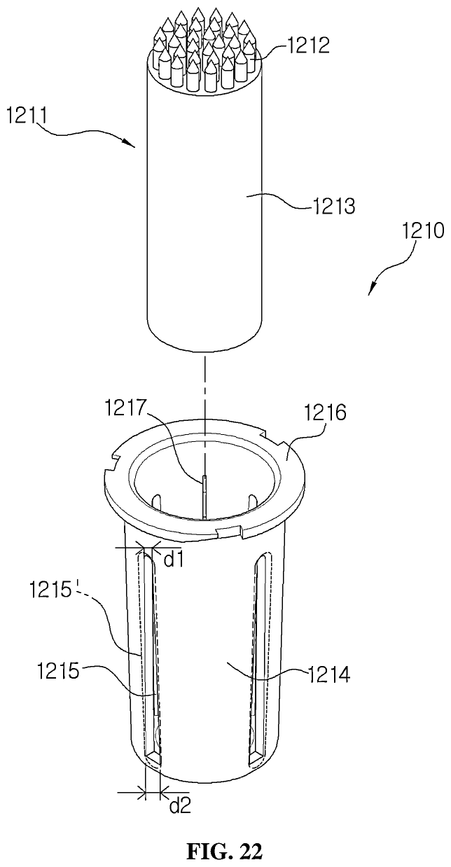

FIG. 22 is an exploded perspective view illustrating a modification example of the base solution absorption electrode module of the sterilization device according to another embodiment of the present disclosure.

Referring to FIG. 22, as another embodiment, the base solution inlet opening part 1215 of the support cover 1214 may have a shape in which only the side surface of the support cover 1214 is open and the opening may not extend to the lower surface of the support cover 1214.

In addition, a liquid-phase base solution inlet cover 1215′ configured to cover the base solution inlet opening part 1215 so as to prevent the liquid-phase base solution stored in the storage casing 1110 to be introduced into the base solution inlet opening part 1215 may be further provided in the base solution inlet opening part 1215.

The liquid-phase base solution inlet cover 1215′ may be positioned inside the support cover 1214 and is coupled to the support cover such that the liquid-phase base solution inlet cover 1215′ is capable of being slid on the support cover 1214 in a circumferential direction, and may optionally open or close the base solution inlet opening part 1215.

Particularly, when the operation of the sterilization device 1000 is stopped by a separate operation switch (not illustrated) or when the liquid-phase base solution stored in the storage casing 1110 is below the lower operating limit L and the operation of the sterilization device 1000 is stopped, the liquid-phase base solution inlet cover 1215′ is slid according to a separate controller (not illustrated), so that the base solution inlet opening part 1215 may be blocked. In addition, even in a state in which the base solution inlet opening part 1215 is blocked, the sterilization device 1000 is operated for a predetermined time so that the liquid-phase base solution absorbed by the absorption member 1211 is removed, so that there is an effect that a contamination of the absorption member 1211 such as mold due to a state in which the absorption member 1211 is submerged in the liquid-phase base solution may be prevented.

In addition, the absorption member 1211 may be formed of an antimicrobial material such as silver ions (Ag+), so that the contamination such as mold may be prevented.

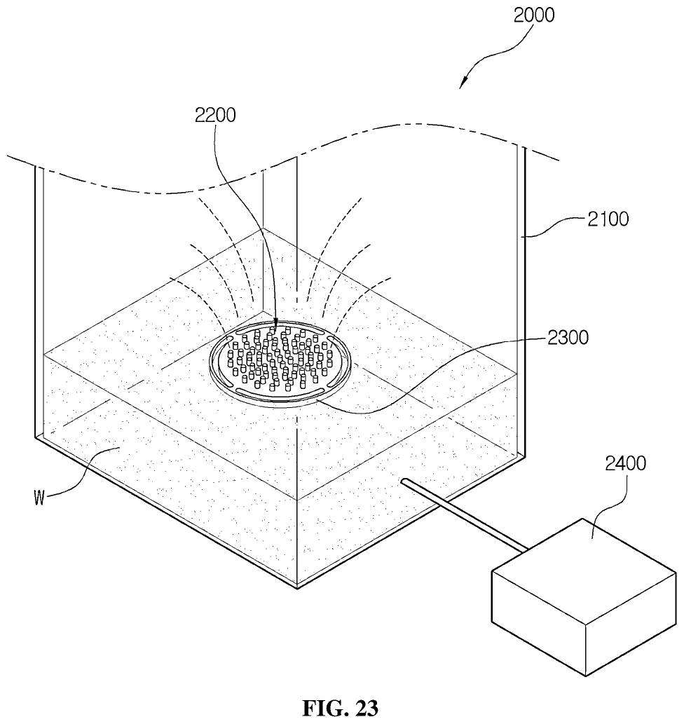

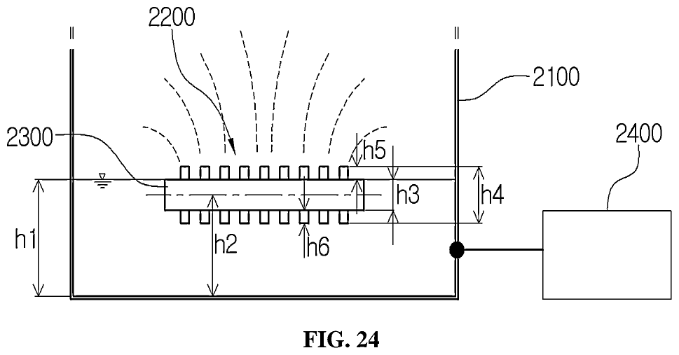

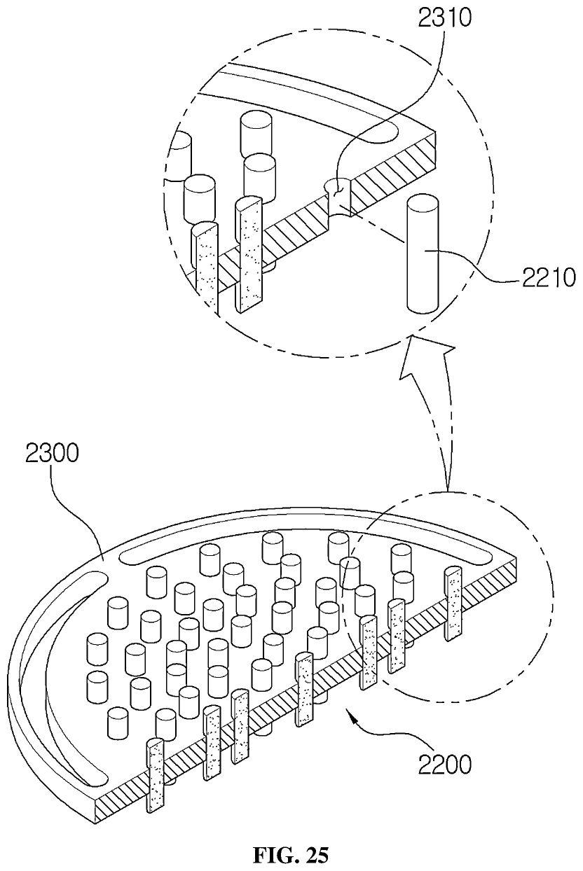

FIG. 23 is a perspective view illustrating the sterilization device according to still another embodiment of the present disclosure, FIG. 24 is a cross-sectional view illustrating the sterilization device according to still another embodiment of the present disclosure, and FIG. 25 is an enlarged cross-sectional view illustrating a floating unit of the sterilization device according to still another embodiment of the present disclosure.

Referring to FIG. 23 to FIG. 25, a sterilization device 2000 according to still another embodiment of the present disclosure includes: an electrostatic spray unit 2200 configured to convert the base solution stored in a housing 2100 that stores the liquid-phase base solution W into a discharge solution having a sterilization function using hydroxide ions; and a height adjustment unit 2300 provided inside the housing 2100 and configured to automatically adjust a height of the electrostatic spray unit 2200 according to a storage boundary height of the base solution W.

The height adjustment unit 2300 is a floating unit 2300 which floats on the base solution W and which is adjusted. Furthermore, the floating unit 2300 may further include a discharge guide unit (not illustrated) configured to absorb the base solution W, the discharge guide unit being configured such that the discharge solution having the sterilization function using hydroxide ions (OH⁻) is formed by using the base solution W, and the discharge guide unit being configured to guide the discharge solution to be discharged to the outside of the housing 2100.

The floating unit 2300 is formed of a material having a specific gravity smaller than that of the base solution W stored in the housing 2100, so that an end portion of the electrostatic spray unit 2200 may be exposed to the outside of the base solution W.

Specifically, a floating height h2 of the floating unit 2300 may be smaller than the height h1 of the base solution W.

The electrostatic spray unit 2200 may include a fiber part 2210 which is fixed to a through-hole part 2310 provided in the floating unit 2300 and which is configured to discharge the discharge solution to the outside of the housing 2100.

The fiber part 2210 may extend such that the fiber part 2210 has constant heights h5 and h6 upward and downward of the floating unit 2300, and may be exposed to the outside of the floating unit 2300. Particularly, a height h4 of the fiber part 2210 may be larger than a height h3 of the floating unit 2300.

Preferably, the height h5 by which the fiber part 2210 extends upward from the floating unit 2300 may be equal to the height of the fiber part 2210 from the surface of the base solution W.

The fiber part 2210 may be a fiber bundle in which a plurality of unit fibers is coupled to each other.

The unit fiber may be selected from a group consisting of glass fiber, carbon fiber, metal fiber, aramid fiber, polyamide fiber, and a mixture or a combination thereof.

The unit fiber may be selected from a group consisting of a unit fiber having a circular cross-sectional area and a unit fiber having a non-circular cross-sectional area, and may preferably exist in the form of a short-fiber. Particularly, the unit fiber may preferably exist in the form of a stable fiber with a length ranging from 0.1 mm to 50 mm or in the form of a continuous fiber (roving).

A voltage application unit 2400 electrically connected to the electrostatic spray unit 2200 and configured to supply power to the electrostatic spray unit 2200 may further be included in the outside or the inside of the housing 2100.

The sterilization device according to still another embodiment of the present disclosure may include: a housing that stores a liquid-phase base solution therein; and a spraying unit configured to spray a spraying solution in which a plurality of heterogeneous ions carrying an electrical charge by applying a high-voltage to the base solution is included and has an airborne bacteria sterilization power of 90% or more at an air temperature of 25 degrees Celsius and an air velocity of 2 m/s.

Particularly, a sterilization solution according to an embodiment of the present disclosure may include: a plurality of heterogeneous ions carrying an electrical charge; and a spraying solution having an airborne bacteria sterilization power of 90% or more at an air temperature of 25 degrees Celsius and an air velocity of 2 m/s.

This is a result of the ISO 16000-36 standard experiment, and the sterilization solution according to the present disclosure has a sterilization force equal to or more than 90% in the standard of the ISO 16000-36 standard experiment.

The plurality of heterogeneous ions may include at least one ion selected from H⁺, OH⁻, and O₂⁻.

The spraying solution sprayed by embodiments of the present disclosure is generated by performing electrostatic spraying by applying a high-voltage to the base solution, and includes liquid-phase particles and at least one ion selected from H⁺, OH⁻, and O₂⁻, reacts with simultaneously generated electrons to produce at least one ion selected from H⁺, OH⁻, and O₂⁻ and H₂O₂ and H₂O, and finally forms a large amount of OH⁻.

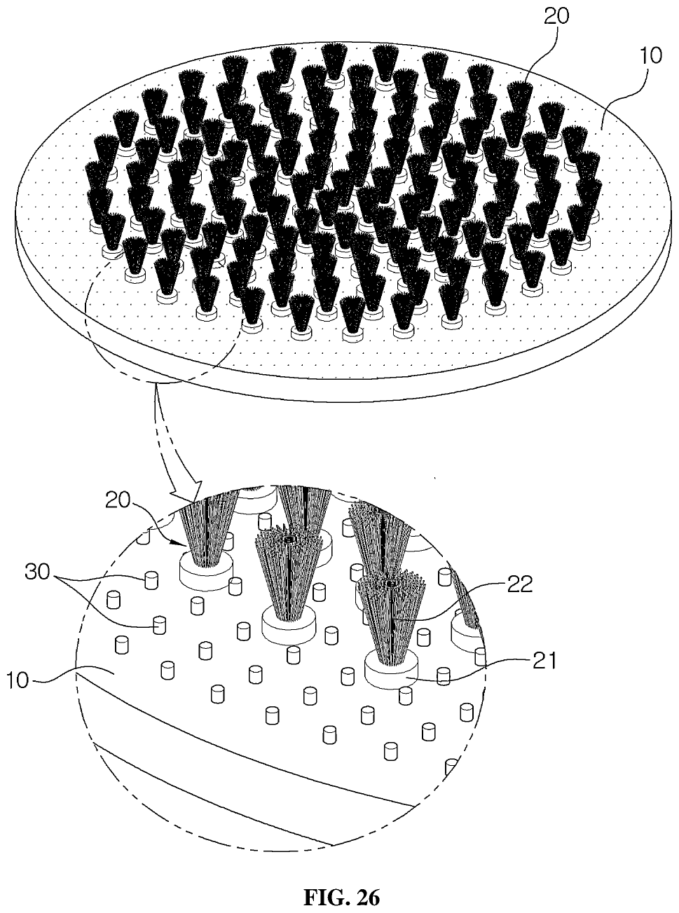

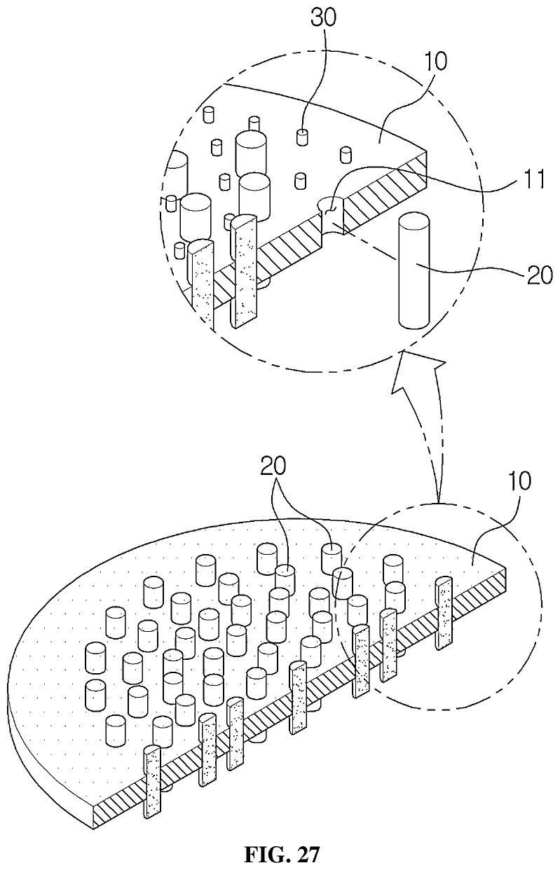

Meanwhile, FIG. 26 is a perspective view schematically illustrating an array module for electrostatic spraying according to an embodiment of the present disclosure. FIG. 27 is a cross-sectional perspective view illustrating the array module for electrostatic spraying in FIG. 26.

Referring to FIG. 26, the array module for electrostatic spraying according to an embodiment of the present disclosure is used in an electrostatic atomization-type electrostatic spray system configured to generate and spray a base solution in the form of micro-sized droplets through electrostatic induction with an electrode (not shown) when a voltage is applied. Furthermore, the array module for electrostatic spraying is disposed adjacent to a liquid-phase base solution such that the array module for electrostatic spraying faces and is spaced apart from an electrode to which power is applied, and may include a plate 10 in which at least one coupling hole 11 is formed and may include at least one nozzle 20 mounted in the plate 10.

As illustrated in FIG. 26, the plate 10 may be formed as a plate-shaped member. At least one coupling hole 11 may be formed through the plate 10. The coupling hole 11 formed in the plate 10 may be formed as a plurality of coupling holes 11 formed in a matrix structure across a surface of the plate 10. In the present embodiment, the plate 10 is formed as a circular plate-shaped member, and the coupling holes 11 is formed in the surface of the plate 10 and forms a circular arrangement. In contrast, although not illustrated in the drawings, the plate 10 may be formed as a rectangular plate-shaped member, and the shape of the plate 10 may be variously modified according to the type and the size of the device used.

As illustrated in FIG. 26 and FIG. 27, the nozzle 20 may be mounted in the plate 10 by being coupled to the coupling hole 11. The nozzle 20 may be formed of a material having an excellent hydrophilicity or an excellent absorptivity, and may be formed in a small size of a micro level. For example, the nozzle 20, which will be described later, may be provided in the form of a porous member (see FIG. 27) or a fiber bundle (see FIG. 26). Such a nozzle 20 may be provided in a quantity corresponding to each of the coupling holes 11, and may be coupled to each of the coupling holes 11. Here, as illustrated in FIG. 27, the nozzle 20 may be mounted in the plate 10 by simply fitting and inserting the nozzle 20 into an upper side or a lower side of the coupling hole 11 and by fixing the nozzle 20 to the coupling hole 11 by using a fixing member (not illustrated) such as a separate clamp, or may be mounted in the plate 10 by being screw-fastened to the coupling hole 11. Such a nozzle 20 may be provided such that both end potions of the nozzle 20 protrude to the upper surface and the lower surface of the plate 10 respectively.

At this time, in the array module for electrostatic spraying according to an embodiment of the present disclosure, since the nozzle 20 protrudes through a first side surface of the plate 10, electrostatic spraying may be performed by a portion of the nozzle 20 that protrudes through the first side surface. Furthermore, the array module for electrostatic spraying may be configured such that the base solution W below the plate 10 is absorbed by a portion of the nozzle 20 that protrudes through a second side surface of the plate 10 and the base solution W is transferred to the protrusion portion of the nozzle 20 at the first side, or may be configured such that surrounding moisture is absorbed and transferred by the hydrophilic material of the nozzle 20, so that the base solution W is capable of being supplied without a separate transferring device and is capable of being used for electrostatic spraying. That is, when the array module for electrostatic spraying is mounted in a device in which a storage container having a base solution therein is provided, the array module for electrostatic spraying is configured such that the array module for electrostatic spraying absorbs the base solution. Furthermore, when the array module for electrostatic spraying is mounted in a humidifier, an air conditioner, and so on, the array module for electrostatic spraying is configured such that the array module for electrostatic spraying absorbs moisture that exists as a water vapor or a liquid-phase solution, so that fluid required for electrostatic spraying is capable of being supplied by using the array module for electrostatic spraying.

According to a structure that will be described in detail later, in the array module for electrostatic spraying according to an embodiment of the present disclosure, the liquid-phase base solution or the surrounding moisture absorbed through the nozzle 20 is capable of being sprayed in the form of an aerosol in a micro-sized droplet state having the sterilization function by using the electric field formed by applying a voltage applied to the electrode or the plate 10.

As such, in contrast to an existing nozzle in which an end portion of the existing nozzle protrudes toward only one direction intended to spray a solution, the nozzle 20 of the present disclosure has a structure in which the nozzle 20 protrudes through the upper surface and the lower surface of the plate 10. That is, since the nozzle 20 is provided such that the nozzle 20 protrudes through the upper surface and the lower surface of the plate 10, the base solution W is absorbed without using a separate fluid transferring device, and the absorbed base solution W is capable of being sprayed in the electrostatic spraying manner, so that the function of absorbing the base solution W and the function of spraying the base solution W are capable of being performed simultaneously.

That is, the array module for the electrostatic spraying according to an embodiment of the present disclosure does not require a separate pump or a configuration of an atomization device or a blowing device, so that the manufacturing cost of the array module for electrostatic spraying may be lowered. Furthermore, there are effects that the device is capable of being miniaturized, the device is easy to manage, and the cost in terms of maintenance is reduced.

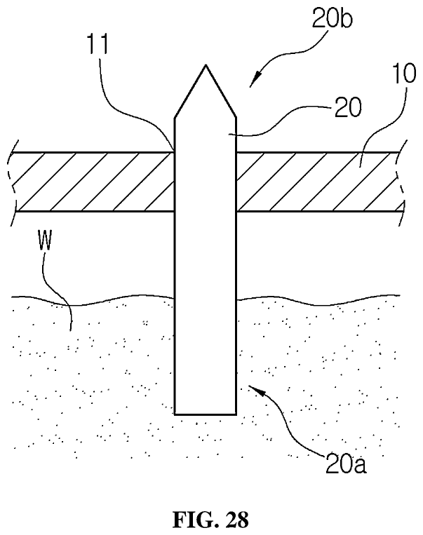

FIG. 28 is a view illustrating a cross-section of the array module for electrostatic spraying according to an embodiment of the present disclosure.

Specifically, according to an embodiment of the present disclosure, the nozzle 20 may include an absorption part 20a and a tip part 20b as illustrated in FIG. 28. Here, the nozzle 20 may be provided in a structure in which the absorption part 20a and the tip part 20b are formed in separate configurations and are integrally connected to each other by mutual coupling. However, in the present embodiment, the absorption part 20a and the tip part 20b may be provided in an integrated configuration in which the absorption part 20a and the tip part 20b extend each other, and may be provided as portions that protrude to the lower surface and the upper surface of the plate 10 respectively.

As illustrated in FIG. 28, the absorption part 20a may protrude through the lower surface of the plate 10, and may be inserted into the liquid-phase base solution W.

As illustrated in FIG. 28, the tip part 20b may extend from the absorption part 20a and may protrude through the upper surface of the plate 10. The tip part 20b may be provided such that the tip part 20b faces an electrode that is positioned apart upward from the tip part 20b.

The nozzle 20 configured as described above will be described later. The nozzle 20 may be formed of an absorbent material having an excellent absorption function so that the absorption part 20a is capable of being immersed by the base solution when the absorption part 20a is inserted into the base solution W. For example, the nozzle 20 may be formed of a porous material. For example, the nozzle 20 may be formed of a polymer or a synthetic resin such as polyethylene, urethane, or EVA. In the nozzle 20, the base solution W absorbed through the absorption part 20a at the lower side of the nozzle by the capillary phenomenon may be moved to the tip part 20b at the upper side of the nozzle 20. Accordingly, as the nozzle 20 absorbs the liquid-phase base solution W in contact with the lower portion of the absorption part 20a, the liquid-phase base solution W is capable of being transferred toward the tip part 20b.

In addition, the absorption part 20a of the nozzle 20 may be formed such that a length of the absorption part 20a is longer than a length of the tip part 20b. That is, the absorption part 20a extends to a storage container (not illustrated) in which the base solution W is accommodated, and may be inserted into the base solution W as the absorption part 20a extends downward from the plate 10. The length of the absorption part 20a may extend to the bottom of the storage container or a position of a lower limit where the base solution is stored.

Accordingly, even when the level of the base solution W is lowered, the nozzle 20 is capable of continuously maintaining the absorbing and transferring state of the base solution W by the absorption part 20a. In contrast, in the present disclosure, even when the absorption part 20a is formed such that the length of the absorption part 20a is short, a state in which the absorption part 20a is in contact with the base solution is capable of being maintained by providing a separate water level adjustment part or a separate height adjustment part.

Meanwhile, since the nozzle 20 may be coupled to the plate 10 by a simple fitting, a fixing method by a clamp, or a screw-fastening method, the lengths of the absorption part 20a and the tip part 20b may be adjusted differently by adjusting the position of the nozzle 20 fixed to the plate 10. In contrast, the nozzle 20 may adjust the length of the absorption part 20a by connecting a separate extension part (not illustrated) for extending the length of the absorption part 20a to the end portion of the absorption part 20a. In addition, a separate adjustment mechanism (not illustrated) for adjusting the height of the plate 10 may be provided. For example, the plate 10 may be disposed in a state in which the plate 10 floats on the base solution W, and may be moved together as the level of the base solution W increases or decreases.

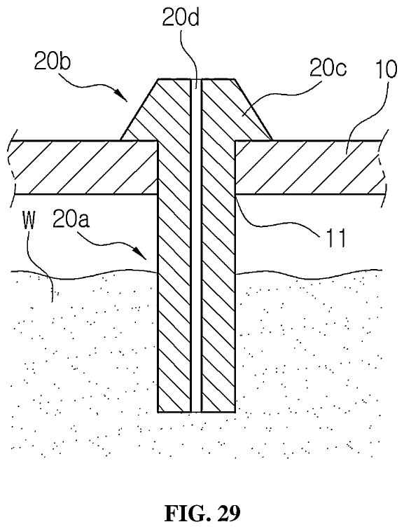

FIG. 29 is a cross-sectional view illustrating a modification example of the array module for electrostatic spraying according to an embodiment of the present disclosure.

Referring to FIG. 29, the nozzle 20 may further include a cone part 20c.

The cone part 20c may protrude to the outside of the tip part 20b. As illustrated in FIG. 29, the cone part 20c may extend from the tip part 20b, and may extend obliquely outward in a radial direction toward the surface of the plate 10 from a distal end of the tip part 20b. Accordingly, by the cone part 20c, the nozzle 20 may have a substantially conical shape in which the shape formed by the tip part 20b is wider at the bottom and narrower at the top. For example, the nozzle 20 may be formed in a conical shape or a polygonal horn shape.

Such a cone part 20c has a conical structure, and a crosstalk phenomenon with the adjacent nozzle 20 may be minimized as the thickness of the cone part 20c is gradually reduced from a portion of the cone part 20c in contact with the surface of the plate 10 to the upper end portion of the tip part 20b.

That is, due to the conical structure, a large amount of electrostatic spraying operation is capable of being performed by minimizing interference that occurs between adjacent nozzles 20 when an electrostatic charge is generated.

Accordingly, since the array module for the electrostatic spraying of the present disclosure is capable of stably spraying a large amount of aerosols, the efficiency of the electrostatic spraying may be increased.

Furthermore, as illustrated in FIG. 29, the nozzle 20 may further include a hole part 20d.

The hole part 20d may be formed as a hole that penetrates the nozzle 20, and may be provided such that the hole part 20d is in communication with the coupling hole 11 of the plate 10.

Accordingly, according to the present disclosure, the hole part 20d may further increase the absorption action of the base solution W by the nozzle 20. Furthermore, as the hole size of the end portion of the tip part 20b has a small size, the aerosol generated by the nozzle 20 may be further be micronized.

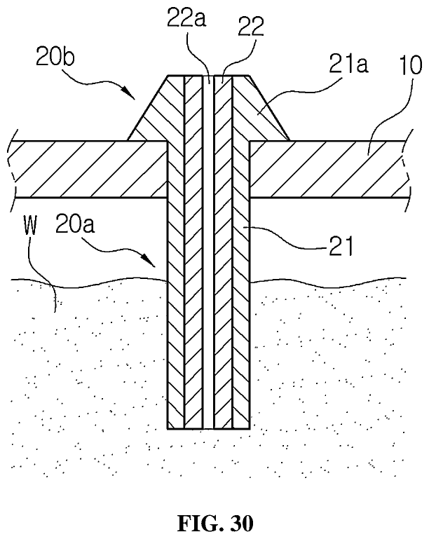

FIG. 30 is a cross-sectional view schematically illustrating the nozzle for electrostatic spraying according to another embodiment of the present disclosure.

According to another embodiment of the present disclosure, as illustrated in FIG. 30, the nozzle 20 may include a support tube 21 and an absorption member 22.

As illustrated in FIG. 30, the support tube 21 may be formed as a tubular member, may be provided as at least one member, and may be coupled to each coupling hole 11 of the plate 10. Such a support tube 21 may protrude to the upper surface and the lower surface of the plate 10. The support tube 21 may be connected to the coupling hole 11 in a manner in which the nozzle 20 is coupled to the plate 10. That is, the support tube 21 is coupled to the coupling hole 11 and is fixed to the plate 10 by a separate clamp or a screw-fastening method.

Here, the support tube 21 may have a cone head part 21a, similar to the way the upper end portion of the aforementioned nozzle 20 is formed in the conical shape. Since the actions and effects caused by the conical shape are the same as those of the embodiment in FIG. 29, the detailed description thereof will be omitted.

As illustrated in FIG. 30, the absorption member 22 may be inserted into and fixed to the inside of the support tube 21. The absorption member 22 is a member that simultaneously performs the function of the absorption part 20a and the function of the tip part 20b, and may be provided such that both end portions of the absorption member 22 extend to both end portions of the support tube 21. The absorption member 22 may extend such that the end portion of the absorption member 22 is submerged in the base solution W. As described above, the absorption member 22 may be submerged in the base solution since the absorption member 22 is formed such that the portion of the absorption member 22 having the function of the absorption part 20a has a long length. On the other hand, the nozzle 20 may be formed such that the portion of the nozzle 20 having the function of the absorption member 22 or the function of the support tube 21 that protrudes to the lower surface of the plate 10 has a long length.

Here, a penetration hole 22a may be formed in the absorption member 22, and may be provided such that the penetration hole 22a is in communication with the coupling hole 11 of the plate 10. Such a penetration hole 22a may further increase the absorption action of the base solution W by the nozzle 20. Furthermore, as the hole size of the upper end portion of the absorption member 22 has a small size, the droplets generated by the nozzle 20 may be further be micronized.

As such, since the nozzle 20 is divided into the support tube 21 and the absorption member 22, the support tube 21 and the absorption member 22 may be separately provided.

That is, the nozzle 20 may be easily replaced when the absorption member 22 is replaced with a member of a different material as required. Furthermore, the nozzle 20 may be easily replaced when the nozzle 20 is required to be replaced due to performance degradation or damage.

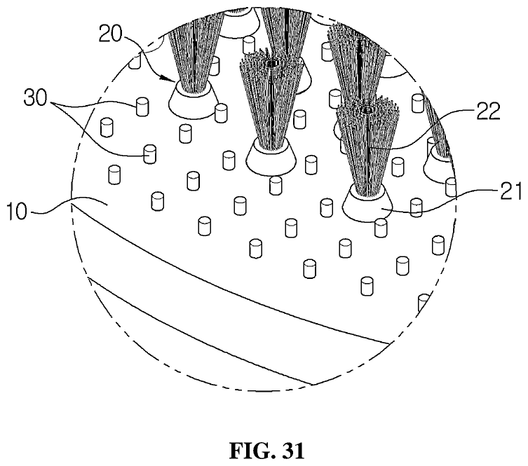

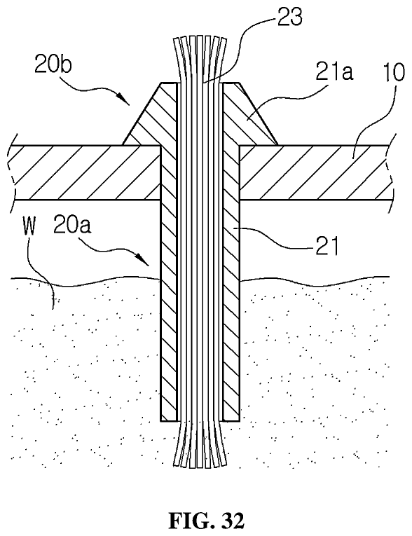

Meanwhile, FIG. 31 and FIG. 32 are views illustrating the array module for electrostatic spraying according to still another embodiment of the present disclosure.

According to an embodiment of the present disclosure, the absorption member 22 may be formed of a porous material having excellent absorptivity as described above in the embodiment in FIG. 28, but may be formed of a fiber material as another example.

Specifically, as illustrated in FIG. 31 and FIG. 32, the absorption member 22 may be formed of a fiber bundle 23 formed of a plurality of fibers. The absorption member 22 formed of the fiber bundle 23 may be inserted into and fixed to the support tube 21. Such a fiber bundle 23 may be inserted into the support tube 21 in a state in which the plurality of fibers is bound as a bundle, and may be easily withdrawn and replaced.

Here, the fiber bundle 23 may be formed of carbon fiber. The fiber bundle 23 formed of carbon fiber is bound in a state in which the fiber bundles 23 are in contact with each other. However, when a voltage is applied to the fiber bundle 23, each of the fiber bundles 23 is charged to the same polarity, so that a repulsive force may be generated between end portions of each of the fiber bundles 23. Accordingly, when a voltage is applied to the fiber bundle 23 that is bound as one bundle, a repulsive force pushing the end portion of each of the fiber bundles 23 such that the end portion of each of the fiber bundles 23 is spaced apart from each other occurs, and the fiber bundle 23 is spread in a radial direction, so that the micro droplets are capable of being sprayed in the scattered state.

As another embodiment, although not illustrated, the absorption member 22 described above may be formed by coupling a plurality of unit fibers. That is, the unit fiber may be selected from a group consisting of glass fiber, carbon fiber, metal fiber, aramid fiber, polyamide fiber, and a mixture or a combination thereof. Such a unit fiber may be selected from a group consisting of a unit fiber having a circular cross-sectional area and a unit fiber having a non-circular cross-sectional area, and may preferably exist in the form of a short-fiber. Particularly, the unit fiber may preferably exist in the form of a stable fiber with a length ranging from 0.1 mm to 50 mm or in the form of a continuous fiber (roving).

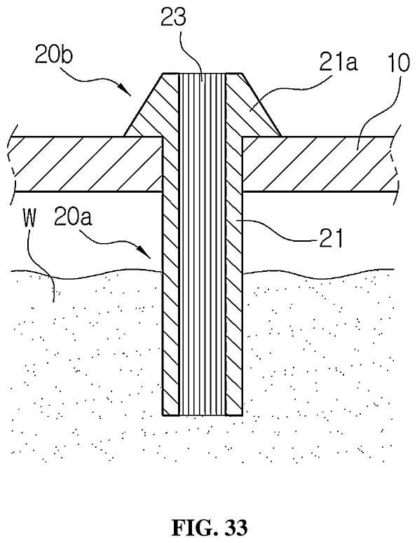

FIG. 33 is another modification example of the array module for electrostatic spraying according to an embodiment of the present disclosure illustrated in FIG. 32.

According to the present embodiment, as illustrated in FIG. 33, the nozzle 20 may be configured in a form in which both end portions of the fiber bundle 23 are cut off at positions corresponding to each end portion of the support tube 21. That is, the fiber bundle 23 may be provided such that the fiber bundle 23 is exposed to the outside by the penetration hole of the support tube 21 and does not protrude from the end portion of the support tube 21.

Accordingly, the fiber bundle 23 that protrudes toward the upper end of the nozzle 20 does not extend outward, so that interference with the adjacent nozzles may be minimized. Since interference with the adjacent nozzles is minimized, aerosols may be sprayed stably, so that spray efficiency may be increased.

Meanwhile, a communication hole (not illustrated) allowing entering and exiting of the base solution W so that the liquid-phase base solution W is introduced and absorbed into the absorption member 22 may be formed in the support tube 21. The communication hole (not illustrated) may be provided as a hole that penetrates a side direction of the support tube 21, or may be provided in a slit shape formed along a longitudinal direction of the support tube 21. Such the communication hole (not illustrated) allows the base solution W to be introduced into the tube of the support tube 21, thereby increasing the absorption action by the absorption member 22. In addition, when the communication hole (not illustrated) is provided as a long hole-type slit, the introduction amount of the base solution W may be further increased, and the contact area of the absorption member 22 with respect to the base solution W may expand simultaneously.

That is, in the nozzle 20, due to the shape in which the both ends of the support tube 21 are open, the base solution W is capable of being absorbed by the absorption member 22 that protrudes at the lower end of the nozzle 20. Furthermore, by using the communication hole (not illustrated), the base solution W is capable of being additionally introduced into the support tube 21, so that the rapid absorption performed by the absorption member 22 may be induced.

Accordingly, even when the nozzle 20 has a long length, the base solution W may be rapidly absorbed, and the time of transferring the base solution W to the opposite end portion of the nozzle 20 may be shortened.

That is, according to an embodiment of the present disclosure, since the base solution contact area of the absorption member 22 is increased, the absorption storage amount of the liquid-phase base solution is increased and the spraying amount of the aerosol sprayed from the end portion of the nozzle 20 is increased, and the spraying of the aerosol from each of the nozzles 20 is stably performed, so that there is an effect that the operation stability is increased.

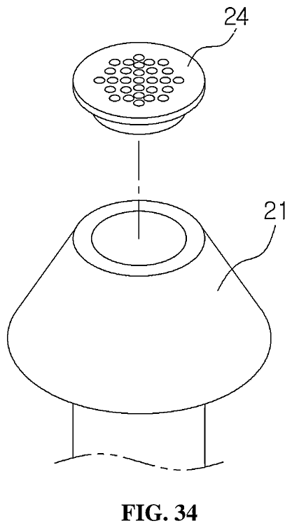

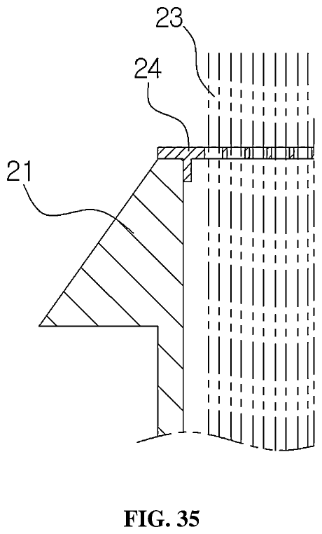

FIG. 34 to FIG. 38 are views respectively illustrating the array module for electrostatic spraying according to other embodiments of the present disclosure.

Referring to FIG. 34 to FIG. 36, the nozzle 20 according to an embodiment of the present disclosure may further include a porous cap 24.

The porous cap 24 may be formed as a porous cover member in which a plurality of holes through which fibers enter and exit is formed. Such a porous cap 24 may be fitted and coupled to the end portion of the support tube 21. Accordingly, the porous cap 24 may be configured such that each fiber contained in the fiber bundle 23 is partitioned as a single fiber or is partitioned in a state in which each fiber contained in the fiber bundle 23 is partitioned into at least two bundles. That is, the fibers of the fiber bundle 23 may be inserted into the porous cap 24 through the plurality of holes, and may maintain the fibers contained the fiber bundle 23 such that the fibers are spaced apart from each other and are maintained in a single group or a plurality of groups. Accordingly, as a gap is provided by positioning the fibers of the fiber bundle 23 to be spaced apart from each other by a predetermined distance in the support tube 21 by the porous cap 24, the contact area of the base solution W is increased, and the fibers absorb the base solution W and the base solution W is capable of being transferred through the space between the fibers. That is, the capillary shape may be increased.

At this time, the porous cap 24 may include a first cap member 24a and a second cap member 24b. The first cap member 24a may be coupled to the first end of the support tube 21, i.e., the upper end of the support tube 21. Furthermore, the second cap member 24b may be coupled to the lower end of the support tube 21. Therefore, since the porous cap 24 is configured such that the porous cap 24 holds the fiber bundle 23 at both ends of the support tube 21, stable fixing is capable of being realized.

At this time, the position of the fiber bundle 23 passing through the first cap member 24a and the position of the fiber bundle 23 passing through the second cap member 24b may be different from each other. That is, the fiber bundle 23 is formed in a shape in which the fiber bundle 23 is twisted along a first direction in a wire screw manner, and the position into which the fiber bundle 23 is inserted into the first cap member 24a and the position through which the fiber bundle 23 passes the second cap member 24b may be different from each other. Therefore, the nozzle 20 may increase the absorption action of the base solution W due to the twisted structure of the fiber bundle 23.

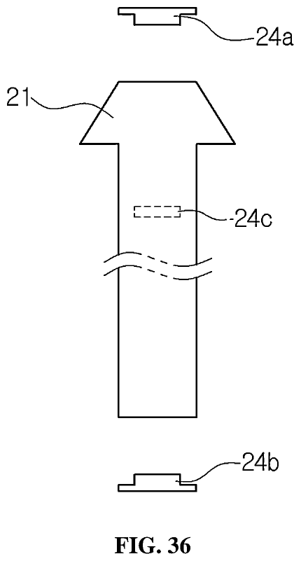

In addition, as illustrated in FIG. 36, the nozzle 20 may further include a gap maintaining member 24c disposed between the first cap member 24a and the second cap member 24b.

The gap maintaining member 24c may be disposed such that the gap maintaining member 24c is spaced apart from the first cap member 24a and the second cap member 24b, and may be configured such that the gap maintaining member 24c is inserted into the support tube 21 and holds the fiber bundle 23. The gap maintaining member 24c may be formed of at least one member. Furthermore, the gap maintaining member 24c may be formed as a porous plate that is the same as the porous cap 24, or may be formed as a ring-shaped member. Since the fiber bundle 23 disposed such that the fibers are spaced apart from each other by the porous cap 24 is provided as a member having long length, the gap maintaining member 24c is capable of maintaining the separation space between the fibers of the fiber bundle 23 by supporting the fiber bundle 23 at a middle portion of the support tube 21.

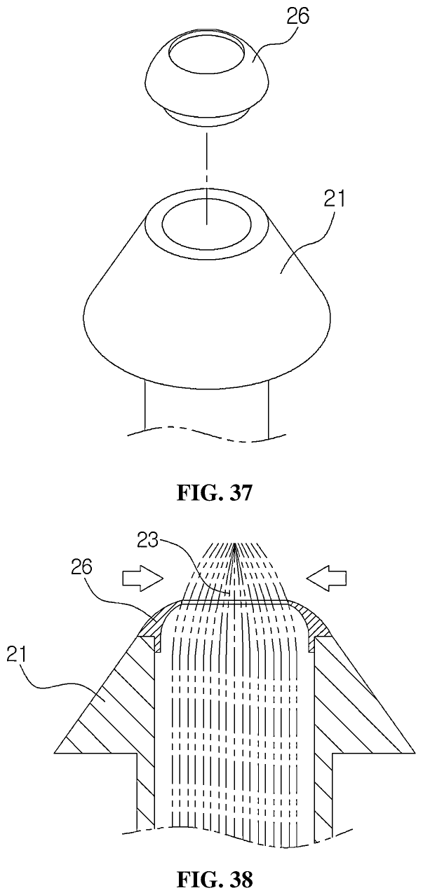

Furthermore, as illustrated in FIG. 37 and FIG. 38. the nozzle 20 may further include an expansion prevention cover 26.

The expansion prevention cover 26 may be provided such that the expansion prevention cover 26 is coupled to a first end of the support tube 21 and supports the fiber bundle 23. The expansion prevention cover 26 may be formed as a conical shape member or a dome shape member having both sides thereof open. The expansion prevention cover 26 is formed in a shape which is wider at the bottom and narrower at the top and which extends upward from the first side end of the support tube 21, and may press the end portion of the fiber bundle 23 from the outside toward the inside. Particularly, as the length of the end portion of the fiber bundle 23 that protrudes toward the first end of the support tube 21 increases, the more the splayed shape is formed, so that the expansion prevention cover 26 may be formed such that the expansion prevention cover 26 has an appropriate length and may be configured such that the fiber bundle 23 is prevented from being splayed.

Here, the structure of the expansion prevention cover 26 sharply concentrates the end portion of the fiber bundle 23 regardless of the presence or absence of the porous cap 24 described above, so that the fiber bundle 23 may be prevented from being splayed. That is, in the embodiment described above, when a voltage is applied, the end portions of the fibers are radially spread due to the repulsive force generated on the fiber bundle 23. In this situation, there is an advantage that the micro-sized droplets are sprayed in the scattered state. However, a non-radiative spraying form may be required rather than a radial spraying form according to a situation. Therefore, splaying of the tip part 20b is capable of being prevented by using the expansion prevention cover 26, and a spraying form having a straightness is capable of being realized.

Meanwhile, referring to FIG. 26 and FIG. 27 again, the array module for electrostatic spraying according to an embodiment of the present disclosure may further include a micro protrusion 30.

The micro protrusion 30 may protrude through the upper surface of the plate 10. The micro protrusion 30 may include a plurality of micro protrusions 30, and the plurality of micro protrusions 30 may be disposed around the coupling holes 11. The micro protrusion 30 may act such that the upper surface of the plate 10 has hydrophobicity by the protrusion shape of the micro protrusion 30. In order to increase the hydrophobic effect, a hydrophobic coating may be performed on the surface of the micro protrusion 30 and the surface of the plate 10. Therefore, as the micro protrusion 30 has hydrophobic properties, condensation by surface tension of the liquid component acts, and the droplets accumulated on the nozzle 20 configured to spray the micro-sized droplets are moved toward the micro protrusion 30, thereby inducing the aerosol spraying by the nozzle 20 to be smoothly performed.

In addition, the micro protrusion 30 may include a first protrusion part (not illustrated) and a second protrusion part (not illustrated). The first protrusion part (not illustrated) may be formed of a group of protrusions spaced apart from each other on the circumference of the coupling hole 11 and distributed adjacent to the nozzle 20. The second protrusion part (not illustrated) may be formed of a group of protrusions spaced apart from each other on a circumference of the first protrusion part (not illustrated) and positioned far from the nozzle 20. As such, since the micro protrusion 30 is configured as the plurality of micro protrusions 30 including the first protrusion part (not illustrated) and the second protrusion part (not illustrated), the micro protrusion 30 acts such that the droplets generated on the nozzle 20 are spaced apart from each other. At this time, the shape of the first protrusion part (not illustrated) and the shape of the second protrusion part (not illustrated) may be different from each other. That is, end portion shapes of the first protrusion part (not illustrated) and the second protrusion part (not illustrated) may be formed differently such as a square column shape, a cylindrical column shape, a spherical shape, a cone shape, and so on, and such a shape difference is effective in a droplet movement action.

In addition, the micro protrusion 30 may be formed such that a length of the first protrusion part (not illustrated) and a length of the second protrusion part (not illustrated) are different from each other, so that the droplets are capable of being rapidly moved from the nozzle 20.

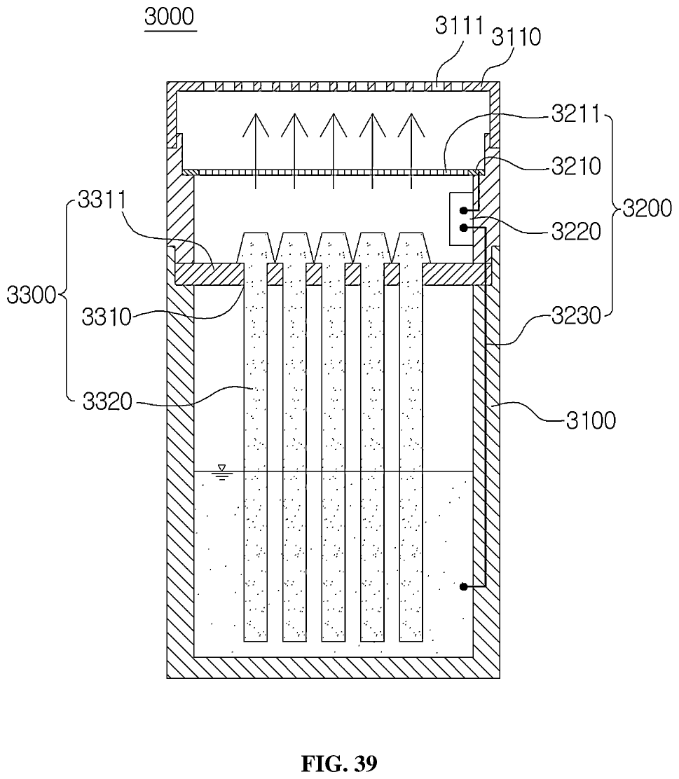

FIG. 39 is a view schematically illustrating the sterilization device according to an embodiment of the present disclosure. The sterilization device according to an embodiment of the present disclosure may include the array module for electrostatic spraying described above.

Referring to FIG. 39, the sterilization device according to an embodiment of the present disclosure may be applied to an ion generation element, an ion generation device, an air conditioning device, and so on configured to spray a discharge solution containing ions into the indoor air. The sterilization device according to an embodiment of the present disclosure may be provided as an independent apparatus, or may be mounted in a device such as an air conditioner and so on. The sterilization device according to an embodiment of the present disclosure is a device for generating aerosols by using an electrostatic spraying method, and may include a housing 3100, an electrode unit 3200, and a spraying unit 3300.

As illustrated in FIG. 39, a space in which the base solution W is accommodated may be formed inside the housing 3100. The housing 3100 may be formed in a container shape or a cylindrical shape, and may be formed such that a side of the housing 3100 facing the surface of the stored base solution W is open. For example, according to the present embodiment, the housing 3100 may be provided as a cylindrical casing with an open upper portion. At this time, the housing 3100 may have a shape in which a first side of the housing 3100 is open. In the present embodiment, as illustrated in FIG. 39, a discharge port 3110 formed in a cover shape may be provided on the open portion of the housing 3100.

The discharge port 3110 may be coupled to the upper portion of the housing 3100, and may be formed in a shape of an expansion tube that radially extends from the upper end of the housing 3100. The discharge port 3110 may guide the movement of the electrostatically sprayed aerosols, so that the aerosols are capable of being dispersed by a guide part that is formed obliquely toward the outside. A plurality of discharge holes 3111 may be formed in such an outlet 3110, and the aerosols may be discharged to the outside through the discharge holes 3111.

In addition, the housing 3100 may be provided with a blower (not illustrated) for circulating air or a filter (not illustrated) for purifying air, may be configured such that fresh air is introduced from the outside and discharged to the outside together with the aerosols, or may be configured to purify the introduced air.

As illustrated in FIG. 39, the electrode unit 3200 may be mounted inside the housing 3100. The electrode unit 3200 may be disposed such that the electrode unit 3200 crosses the inside of the housing 3100 and is spaced apart upward from the surface of the base solution W, and may include a plate-shaped disk 3210 formed of a conductive material.

The disk 3210 may be formed as an electrode plate. A high-voltage is applied to the disk 3210, and an electrostatic spraying action of the base solution W is generated by inducing a potential difference between the disk 3210 and the spraying unit 3300. A plurality of discharge holes 3211 that discharge micro-sized droplets generated by the spraying unit 3300 may be formed in the disk 3210. The micro-sized droplets generated from the base solution W pass through the discharge holes 3211. At this time, each of the discharge holes 3211 may be formed at a position corresponding to each nozzle portion of the spraying unit 3300 to be described later. Alternatively, the disk 3210 may be formed of a porous plate material having a mesh shape.

Here, in the electrode unit 3200, a high-voltage may be applied to the disk 3210 by a power supply mechanism 3220 mounted inside or outside the housing 3100, or the disk 3210 may act as a ground electrode. That is, in the sterilization device according to an embodiment of the present disclosure, when a voltage is applied to the electrode unit 3200, the spraying unit 3300 may serve as a ground terminal. Furthermore, when the electrode unit 3200 serves as a ground terminal, a voltage may be applied to the spraying unit 3300.

At this time, the power supply mechanism 3220 may include a connection electrode 3230 connected to the base solution W. As the connection electrode 3230 is in contact with the base solution W, power may be applied to the base solution W or a ground power may be connected to the base solution W.

The spraying unit 3300 is an array module for electrostatic spraying described above, is mounted inside the housing 3100, and may serve to spray the base solution W by converting the base solution W into aerosols by using a potential difference with the electrode unit 3200. As illustrated in FIG. 39, the spraying unit 3300 may be disposed such that the spraying unit is spaced apart from the electrode unit 3200, and may be mounted inside the housing 3100 such that a portion of the spraying unit is inserted into the base solution W. As the spraying unit 3300 is in contact with the base solution W, power is capable of being applied to the base solution W from the power supply mechanism 3220 or a ground power may be connected to the base solution W.

In addition, the spraying unit 3300 may include a base plate 3310 and a spraying nozzle 3320. Since these configurations may be configured in the same configuration as the array module for electrostatic spraying according to the embodiment described above, the detailed description of these configurations will be omitted.

As illustrated in FIG. 39, the base plate 3310 may be formed as a plate-shaped member, and at least one coupling hole 311 may be formed through the base plate 3310. Such a base plate 3310 is fixed to the inside of the housing 3100 by being spaced apart from the disk 3210 of the electrode unit 3200 to which power is applied, and may be disposed adjacent to the liquid-phase base solution W.

As illustrated in FIG. 39, the spraying nozzle 3320 may be mounted in the base plate 3310 by being fitted to the coupling hole 311, and may be provided such that both end portions of the spraying nozzle 3320 protrude through an upper surface and a lower surface of the base plate 3310.

The spraying unit 3300 is capable of spraying the liquid-phase base solution W absorbed through the spraying nozzle 3320 such that the aerosols in the micro-sized droplet state having the sterilization function through the electric field generated by applying a voltage to the plate 3310 are sprayed.

In the sterilization device according to an embodiment of the present disclosure, the spraying nozzle 3320 absorbs the base solution W without using a separate fluid transferring device, is provided such that the spraying nozzle 3320 protrudes through the upper surface and the lower surface of the base plate 3310 so that the absorbed base-solution W is sprayed in the electrostatic spraying manner, so that the function of absorbing the base solution W and the function of spraying the base solution W may be performed simultaneously.

That is, the sterilization device according to an embodiment of the present disclosure does not require a configuration of an atomization device or a blowing device, so that the manufacturing cost of the sterilization device may be lowered. Furthermore, there are effects that the device is capable of being miniaturized, the device is easy to manage, and the cost in terms of maintenance is reduced.

The spraying unit 3300 serves to absorb the liquid-phase base solution W and to spray the base solution W by using the potential difference with the electrode unit 3200 such that the aerosols are sprayed.

As such, the electrode unit 3200 and the spraying unit 3300 may perform a function of converting the liquid-phase base solution W stored in the housing 3100 into the aerosols having the sterilization function using hydroxide ions (OH⁻) and then spraying the aerosols.

Specifically, in the sterilization device according to an embodiment of the present disclosure, O₂⁻(H₂O)n (where n is any natural number) as minus ions and H⁺(H₂O)m (where m is any natural number) as plus ions may be generated. In addition, by transferring these ions into air, the sterilization device induces an oxidation reaction by hydrogen peroxide H₂O₂ or OH radicals as an active species generated by a chemical reaction of these ions, so that bacteria floating in the air may be sterilized.

In the sterilization device according to an embodiment of the present disclosure as described above, the sterilization device may convert the liquid-phase base solution W stored in the housing 3100 into the aerosols in the nano-sized droplet state having a high charge by using the electric field generated by the electrode unit 3200 and the spraying unit 3300, and may spray the aerosols forming hydroxide ions (OH⁻) by using the electric field generated due to the voltage difference.

As such, in the sterilization device, since moisture contained in the aerosols is ionized by the electric field, hydroxide ions (OH⁻) are distributed on the surfaces of the aerosols. Since such hydroxide ions perform the sterilization function, the aerosols generated according to an embodiment of the present disclosure have the sterilization function. Therefore, when the aerosols are discharged to the indoor space, the sterilization function is performed for the corresponding indoor space.

According to such a structure, in the sterilization device according to an embodiment of the present disclosure, the spraying unit 3300 absorbs the liquid-phase base solution W stored in the internal space of the housing 3100, and the aerosols are sprayed by the electric field, so that moisture is sprayed and discharged by the electric field such that the aerosols having hydroxide ions are sprayed and discharged, thereby performing the sterilization function.

Therefore, without using a separate deodorant or a separate air conditioner, the liquid-phase base solution is absorbed and the ionized aerosols are sprayed, so that additional contamination of the device is prevented and an irritating strong smell is not generated. Furthermore, since a periodic operation by the user is not required, there is an advantage that cleaner, hygienic, and convenient use is capable of being realized.

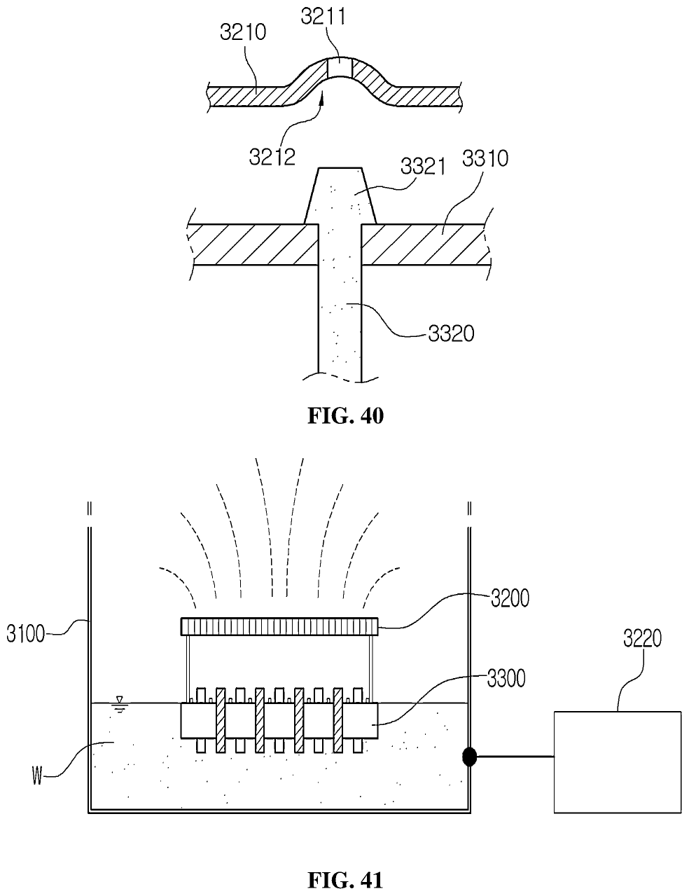

FIG. 40 is a view illustrating a modification example of the sterilization device according to an embodiment of the present disclosure.

Referring to FIG. 40, the disk 3210 of the electrode unit 3200 is spaced apart upward from the plate 3310, and a concave groove part 3212 that protrudes or dents upward in a concave shape centered on the discharge hole 3211 may be formed in the disk 3210.

As described above, due to the concave groove part 3212 that is curved on the surface of the disk 3210, the opposite region of the disk 3210 that has an electric field influence on the tip part of the nozzle 3320 is relatively increased compared to that of a flat plate configuration, so that the intensity of the electric field concentrated on the nozzle 3320 is further increased.

Therefore, the spraying amount of the aerosols sprayed from the nozzle 3320 is increased, and the intensity of the electric field is more concentrated, so that the particle size of the aerosols may be more micronized.

Since the size of the aerosols sprayed in this manner is very fine at the nano-level, and the aerosols are not affected by the weight thereof, the suspension time is increased and, accordingly, the falling amount of the aerosols due to the weight thereof is significantly reduced, so that the external discharge amount of the aerosols are increased to the maximum without waste.

FIG. 41 is a view illustrating another modification example of the sterilization device in FIG. 40.

Referring to FIG. 41, the sterilization device according to an embodiment of the present disclosure may be disposed such that the spraying unit 3300 floats on the base solution W accommodated in the housing 3100. In addition, for the power supply, the power supply mechanism 3220 may be mounted on the outside of the housing 3100 and may be electrically connected to the housing 3100.

At this time, the spraying unit 3300 may be formed of a material having a specific gravity smaller than that of the base solution W in the housing 3100, so that the spraying unit 3300 may float on the base solution W. Furthermore, as the spraying unit 3300 is formed such that the spraying nozzle protrudes through the upper surface and the lower surface of the base plate, the second side of the spraying nozzle may be exposed to the outside of the base solution W while the first side of the spraying nozzle is in contact with the base solution W.

Since the spraying unit 3300 exists in a state in which the spraying unit 3300 floats on the base solution W, a state in which the spraying unit 3300 is in contact with the base solution W may be maintained even when the level of the base solution W is changed due to the use of the base solution W, and the supply of the base solution W may be continuously performed.

Here, in the sterilization device of the present disclosure, a support rod for maintaining a predetermined distance may be mounted between the spraying unit 3300 and the electrode unit 3200. Therefore, as the electrode unit 3200 is moved together with the spraying unit 3300 and the predetermined distance between the electrode unit 3200 and the spraying unit 3300 is maintained constantly, an appropriate electrostatic induction reaction may be generated.

That is, according to an embodiment of the present disclosure, even when the spraying unit 3300 is formed such that the length of the spraying nozzle of the spraying unit 3300 is not long, the supply of the base solution is capable of being smoothly performed due to the floating structure, and the present disclosure is advantageous in simplifying the device since a mechanism for controlling the distance with the base solution W is not separately required.

Although the embodiments of the present disclosure have been disclosed for illustrative purposes, those skilled in the art will appreciate that various modifications, additions, and substitutions are possible, without departing from the scope and spirit of the present disclosure. Accordingly, embodiments disclosed in the present disclosure are provided for describing the present disclosure and are not intended to limit the technical ideas of the present disclosure. The technical ideas of the present disclosure are not limited to the embodiments.

### DESCRIPTION OF REFERENCE NUMERALS

10: Plate
11: Coupling hole
20: Nozzle
20a: Absorption part
20b: tip part
20c: Cone part
20d: Hole part
21: Support tube
22: Absorption member
23: Fiber bundle
24: Porous cap
25: Gap maintaining member
26: Expansion prevention cover
30: Micro protrusion
100, 200, 300, 400: Sterilization device
110: Housing
115: Lid
117: Atomization unit
118: Electrostatic spray unit
120: Discharge guide unit
121, 310, 410: Dispersion tube part
123: Shielding part
127, 414: Support plate 133,
210: Guide tube part
135: Diffusion guide part
138: Retainer
141: Support stand
142: Spoke
143: Hub
147: Discharge tube part
150: Discharge guide
153: Discharge flow path
155: Electrode module
156: Electrode pin
157: Support body
158: Support boss
160: Electrode plate
163: Voltage supply device
165: Wire
220: Blower
1000, 2000, 3000: Sterilization device

1. A sterilization device comprising:
an electrostatic spray unit configured to convert a liquid-phase base solution stored in a housing that stores the base solution into a discharge solution having a sterilization function using hydroxide ions; and
a discharge guide unit coupled to a first side of the housing and provided with a discharge flow path that guides the discharge solution converted by the electrostatic spray unit to be discharged outside the housing.

2. The sterilization device of claim 1, further comprising:
an atomization unit provided inside the housing and configured to generate the discharge solution by atomizing the base solution,
wherein the electrostatic spray unit is positioned above or below the atomization unit, and is configured to convert the discharge solution generated by the atomization unit into a micro-sized droplet state having a high charge.

3. The sterilization device of claim 1, wherein the electrostatic spray unit comprises:
a first electrode module provided inside the housing and provided with a plurality of discharge holes through which the discharge solution is discharged; and
a second electrode module disposed such that the second electrode module is spaced apart from the first electrode module, the second electrode module being configured to transfer the liquid-phase base solution stored inside the housing to a position corresponding to the plurality of discharge holes of the first electrode module and to convert the base solution into the discharge solution by a potential difference generated between the first electrode module and the second electrode module.

4. A sterilization device comprising:
an electrostatic spray unit configured to convert a liquid-phase base solution stored in a housing that stores the base solution into a discharge solution having a sterilization function using hydroxide ions; and
a height adjustment unit provided inside the housing, the height adjustment unit being configured such that a height of the electrostatic spray unit is automatically adjusted according to a storage boundary height of the base solution, wherein the height adjustment unit for adjusting the height of the electrostatic spray unit is a floating unit which floats on the base solution and which is adjusted by the base solution,
wherein the floating unit further comprises a discharge guide unit configured to absorb the base solution and to convert the base solution into the discharge solution having the sterilization function using hydroxide ions (OH⁻), the discharge guide unit being configured to guide the discharge solution to be discharged outside the housing.

5. An array module for electrostatic spraying configured to convert a liquid-phase base solution into micro-sized droplets by generating static electricity between electrodes as power is applied to the array module, the array module comprising:
a plate formed in a plate shape and provided with at least one coupling hole; and
at least one nozzle coupled to the coupling hole and provided such that both end portions of the nozzle protrude through an upper surface and a lower surface of the plate.

6. The array module of claim 5, wherein the nozzle comprises:
an absorption part which protrudes through the lower surface of the plate and which is immersed in the liquid-phase base solution; and
a tip part which extends from the absorption part and which protrudes through the upper surface of the plate.

7. The array module of claim 6, wherein the nozzle further comprises:
a cone part which protrudes on an outer side of the tip part and which extends obliquely and outwardly to the upper surface of the plate from an end portion of the tip part.

8. The array module of claim 5, wherein the nozzle comprises:
at least one support tube which is coupled to the coupling hole and which protrudes through the upper surface and the lower surface of the plate; and
at least one absorption member which is inserted into a tube of the support tube and which has an end portion that extends such that the end portion of the absorption member is immersed in the liquid-phase base solution.

9. The array module of claim 8, wherein the absorption member comprises a fiber bundle in which a plurality of fibers is coupled to each other.

10. The array module of claim 9, wherein the nozzle further comprises:
a porous cap provided on the support tube and configured to partition the fibers included in the fiber bundle such that the fibers are divided in a single state or in a state in which the fibers are divided into two or more fibers.

11. The array module of claim 10, wherein the porous cap of the nozzle comprises a first cap member coupled to a first side end of the support tube and a second cap member which faces the first cap member and which is coupled to a second side end of the support tube, and
wherein the nozzle further comprises a gap maintaining member which is disposed between the first cap member and the second cap member and which is coupled to an inner side of the support tube so as to surround the fiber bundle.

12. The array module of claim 9, wherein the nozzle further comprises an expansion prevention cover which is coupled to a first side end of the support tube and which supports the fiber bundle.

13. The array module of claim 12, wherein the expansion prevention cover is formed in a shape which is wider at a lower side thereof and narrower at an upper side thereof on the first side end of the support tube, and is configured to press an end portion of the fiber bundle from outside.

14. The array module of claim 9, wherein a unit fiber comprising a plurality of unit fibers is capable of being coupled to the fiber bundle, and the unit fiber is selected from a group consisting of glass fiber, carbon fiber, metal fiber, aramid fiber, and a mixture or combination thereof.

15. The array module of claim 5, further comprising:
at least one micro protrusion which is disposed around the coupling hole and which protrudes through the upper surface of the plate.

16. The array module of claim 15, wherein the micro protrusion comprises:
a first protrusion part forming a group which is spaced apart from a circumference of the coupling hole and which is distributed adjacent to the nozzle; and
a second protrusion part forming a group which is spaced apart from a circumference of the first protrusion part and which is distributed at a position far from the nozzle,
wherein a length of the first protrusion part is larger than a length of the second protrusion part.

17. A sterilization device comprising:
a housing in which a liquid-phase base solution is accommodated therein;
an electrode unit spaced apart from a surface of the base solution and mounted inside the housing, the electrode unit being configured to receive a voltage; and
a spraying unit comprising a plate which is disposed such that the plate is spaced apart from the electrode unit and which has at least one coupling hole, the spraying unit comprising at least one nozzle which is coupled to the coupling hole and which protrudes through an upper surface and a lower surface of the plate.

18. The sterilization device of claim 17, wherein the nozzle comprises:
an absorption part which protrudes through the lower surface of the plate and which is immersed in the liquid-phase base solution; and
a tip part which extends from the absorption part and which protrudes through the upper surface of the plate.

19. The sterilization device of claim 18, wherein the nozzle comprises:
a cone part which protrudes on an outer side of the tip part and which extends obliquely and outwardly to the upper surface of the plate from an end portion of the tip part.

20. The sterilization device of claim 17, wherein the nozzle comprises:
at least one support tube which is coupled to the coupling hole and which protrudes through the upper surface and the lower surface of the plate; and
at least one absorption member which is inserted into a tube of the support tube and which has an end portion that extends such that the end portion of the absorption member is immersed in the liquid-phase base solution.
